(19) United States
(12) Patent Application Publication
(10) Pub. No.: US 2025/0367382 A1
(43) Pub. Date: Dec. 4, 2025

(54) MEDICAL INJECTIONS AND RELATED DEVICES AND METHODS

(71) Applicant: Gilead Sciences, Inc., Foster City, CA (US)

(72) Inventors: Walter Goodwin, San Carlos, CA (US); Woo Suk Kim, Hertfordshire (GB); William James Kirby, Cambridge (GB); Finlay Knops-McKim, Cambridge (GB); Daniel Milton Meruz, Cambridge (GB); James Paul Oberhauser, Saratoga, CA (US); Alasdair Young, Foster City, CA (US)

(21) Appl. No.: 19/206,149

(22) Filed: May 13, 2025

Related U.S. Application Data
(60) Provisional application No. 63/647,366, filed on May 14, 2024.

(51) Int. Cl. A61M 5/20; A61M 5/315; A61M 5/32

(52) U.S. Cl. CPC A61M 5/2053; A61M 5/31511; A61M 5/31568; A61M 5/31578; A61M 5/3257; A61M 2005/2013; A61M 2005/206; A61M 2005/208; A61M 2202/0007; A61M 2205/0233; A61M 2205/3317; A61M 2205/3327; A61M 2205/3368; A61M 2205/3523; A61M 2205/502; A61M 2205/581

(57) ABSTRACT

Systems, devices, and methods include an autoinjector including a housing; a connectivity region configured to be accessed at a region of the housing; a needle arranged at a distal end of the housing; a container disposed within the housing and configured to contain medicament; and a plunger slidably disposed within the container. A plunger rod is configured to push the plunger through the container to dispense the medicament through the needle when the container contains medicament. The needle guard is configured to retract when an insertion force is applied to the needle and to extend when the insertion force is removed. A mechanism is configured so that a dispensing movement of the plunger rod, a retraction movement of the needle guard, and/or an extension movement of the needle guard generates an electrical state change. A first sensor is configured to detect the electrical state change.

### CROSS REFERENCE TO RELATED APPLICATIONS

This application claims priority to U.S. Provisional Application No. 63/647,366, filed May 14, 2024, the entire content of which application is hereby incorporated by reference in its entirety.

### TECHNICAL FIELD

This disclosure relates to medical injections and related devices and methods.

### BACKGROUND

An injection typically refers to the act of administering a liquid (e.g., a drug) into a patient's body tissue. Injecting medicament into a patient can allow the medicament to be absorbed relatively rapidly.

### SUMMARY

The present systems, devices, and methods can sense a dispensing movement of a plunger rod within an autoinjector. For example, one or more continuous conductive tracks can be configured to be cut during the dispensing movement, which in turn generates an electrical state change that can be sensed by a sensing assembly to track the dispensing movement of the plunger rod. In turn, the electrical state change can be used to generate an electrical signal, which can include voltage or other electrical measurements.

The devices can include a sensing assembly that can determine that the plunger rod has completed the dispensing movement based on the electrical state change or based on the electrical signal(s). The dispensing movement of the plunger rod corresponds to the amount of medicament delivered to the subject (e.g., a patient). If the plunger rod does not complete the dispensing movement, then the full dose of medicament is not injected into the patient. The sensing assembly can be used to determine that the full dose is injected into the patient by tracking the dispensing movement. The sensing assembly can include a first sensor (e.g., a Near Field Communication (NFC) chip, an NFC coil, or an NFC tag) configured to detect the one or more electrical signals. Such electrical signals can be transmitted from the device to an external device (e.g., a mobile device, smartphone, or tablet) through a wireless communication protocol (e.g., near field communication or others described herein). Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth with a sufficient insertion force and/or whether the needle is removed by removing the insertion force. For example, a needle guard can be configured to retract when an insertion force is applied to the needle and configured to extend when the insertion force is removed. Furthermore, one or more continuous conductive tracks can be configured to be cut during a retraction movement of the needle guard and/or during an extension of the needle guard, which in turn generates an electrical state change that can be sensed by a sensing assembly to track the retraction and extension movements of the needle guard. The electrical state change can be used to generate an electrical signal, which can include voltage or other electrical measurements. The sensing assembly can be used to determine that the full dose is injected into the patient by tracking the retraction movement and the extension movement of the needle guard. Determining dose start and dose end events can be advantageous to ensure that the patient received the medicament properly.

The present disclosure also relates to a composition comprising lenacapavir or a pharmaceutically accepted salt thereof a lenacapavir composition for use in the prevention or treatment of HIV, where the lenacapavir is administered by an autoinjector according to this disclosure. The present disclosure also relates to a use of lenacapavir or a pharmaceutically accepted salt thereof for the manufacture of a medicament for the prevention or treatment of HIV, where the lenacapavir is administered by an autoinjector according to the disclosure.

In a first aspect of the invention, the present disclosure encompasses an autoinjector including a housing; a connectivity region configured to be accessed at a region of the housing; a needle arranged at a distal end of the housing; a container disposed within the housing and configured to contain medicament; a plunger slidably disposed within the container; a plunger rod configured to push the plunger through the container to dispense the medicament through the needle when the container contains medicament; a needle guard configured to retract when an insertion force is applied to the needle and configured to extend when the insertion force is removed; where a dispensing movement of the plunger rod, a retraction movement of the needle guard, and/or an extension movement of the needle guard generates an electrical state change; and a first sensor within the connectivity region configured to detect said electrical state change as the plunger rod and/or the needle guard moves.

According to the first aspect of the invention, the electrical state change may be indicative of dose progression. Where a dispensing movement of the plunger rod generates an electrical state change, the electrical state change may be configured to indicate completion of the dispensing movement of the plunger rod.

According to the first aspect of the invention, the container may contain the medicament. Additionally or alternatively, the medicament may comprise lenacapavir or a pharmaceutically accepted salt thereof.

According to the first aspect of the invention, the autoinjector may further comprise a gas canister assembly configured to release pressurized gas which, when released, provides a force acting on the plunger rod to push the plunger through the container.

According to the first aspect of the invention, the plunger rod includes a first cutter region disposed on a proximal end of the plunger rod. The first cutter region may be configured to cut a first continuous conductive track during the dispensing movement of the plunger rod to provide the first non-continuous conductive track. Optionally, the first continuous conductive track may be provided in proximity to the first cutter region of the plunger rod at an end of the dispensing movement.

According to the first aspect of the invention, additionally or alternatively, the needle guard may be further configured to expose the needle when the insertion force is applied to insert the needle into a user and during the retraction movement of the needle guard, and the retraction movement of the needle guard generates a second electrical state change arising from a second non-continuous conductive track. Optionally, the needle guard may be further configured to cover the needle when the insertion force is removed to withdraw the needle from the user and during the extension movement of the needle guard, and the extension movement of the needle guard generates a third electrical state change arising from a third non-continuous conductive track. Additionally or alternatively, the needle guard may include a second cutter region and/or a third cutter region. The second cutter region may be configured to cut a second continuous conductive track during the retraction movement of the needle guard to provide the second non-continuous conductive track, and/or the third cutter region may be configured to cut a third continuous conductive track during the extension movement of the needle guard to provide the third non-continuous conductive track. Further optionally, the second continuous conductive track may be provided in proximity to the second cutter region of the needle guard at an end of the retraction movement and/or wherein the third continuous conductive track may be provided in proximity to the third cutter region of the needle guard at an end of the extension movement.

The first aspect of the invention may include a transfer sleeve disposed within the housing and configured to interact with the plunger rod and the needle guard, where the first continuous conductive track, the second continuous conductive track, and the third continuous conductive track are disposed on a surface of the transfer sleeve. Additionally or alternatively, the first aspect of the invention may include a linkage disposed within the housing and configured to interact with the transfer sleeve, where the linkage includes one or more features (e.g., guard feature and/or latch feature) configured to enable or prevent cutting of the first continuous conductive track, the second continuous conductive track, and/or the third continuous conductive track disposed on the surface of the transfer sleeve based on positions of the plunger rod and/or the needle guard during or at the end of the dispensing movement and/or the extension movement.

According to the first aspect of the invention, the connectivity region may include one or more passive electrical components. Additionally or alternatively, the first sensor may comprise a Near Field Communication (NFC) chip, an NFC coil, or an NFC tag. Optionally, the connectivity region may be configured to transmit an electrical signal indicative of the electrical state change from the first sensor to a mobile device.

The first aspect of the invention, additionally or alternatively, may include an audible clicker configured to produce one or more audible clicks during the dispensing movement of the plunger rod. Optionally, the audible clicker may include a ring surrounding the plunger rod, and the ring includes a deflectable protrusion configured to contact the plunger rod and produce the one or more audible clicks. Further optionally, the plunger rod may include a ridged surface configured to contact the deflectable protrusion of the audible clicker. Each ridge of the ridged surface may be configured to cause the deflectable protrusion to deflect during the dispensing movement of the plunger rod.

According to the first aspect of the invention, the first sensor may be configured to detect an ambient temperature in proximity to the container.

In a second aspect of the invention, the present disclosure encompasses, an autoinjector including a housing; a needle arranged at a distal end of the housing; a container disposed within the housing and configured to contain medicament; a plunger slidably disposed within the container; a plunger rod configured to push the plunger through the container to dispense the medicament through the needle when the container contains medicament; a needle guard configured to retract when an insertion force is applied to insert the needle into a user and/or configured to extend when the insertion force is removed; a mechanism configured so that a dispensing movement of the plunger rod causes the mechanism to generate a first electrical state change and/or configured so that a retraction movement of the needle guard causes the mechanism to generate a second electrical state change and/or configured so that an extension movement of the needle guard causes the mechanism to generate a third electrical state change; and a first sensor configured to detect said first, second, and/or third electrical state changes generated by the mechanism.

In a third aspect of the invention, the present disclosure encompasses, an autoinjector including a housing; a connectivity region configured to be accessed at a region of the housing; a needle arranged at a distal end of the housing; a needle guard configured to expose the needle when an insertion force is applied to insert the needle into a user; a container disposed within the housing and configured to contain medicament; a plunger slidably disposed within the container; a plunger rod configured to push the plunger through the container to dispense the medicament through the needle when the container contains medicament and when the needle is exposed; and a transfer sleeve including a plurality of continuous conductive tracks, where a movement of the needle guard and/or a movement of the plunger rod generates one or more electrical breaks in at least one of the plurality of continuous conductive tracks.

In a fourth aspect of the invention, the present disclosure encompasses a system including an autoinjector (e.g., any described herein, such as those of the first, second or third aspects of the invention); and a processor configured to process one or more electrical signals to track a dispensing movement of the plunger rod and/or a retraction movement of the needle guard and/or an extension movement of the needle guard.

In a fifth aspect of the invention, the present disclosure encompasses a method of detecting a dispensing movement of a plunger rod within an autoinjector including sensing an electrical state change as the plunger rod moves during the dispensing movement, where the plunger rod includes a region configured to generate one or more electrical breaks in a continuous conductive track, and where the dispensing movement of the plunger rod generates the electrical state change.

In a sixth aspect of the invention, the present disclosure encompasses a method of detecting a retraction movement of a needle guard within an autoinjector including sensing an electrical state change as the needle guard retracts when an insertion force is applied to insert a needle into a user, where the needle guard includes a region configured to generate one or more electrical breaks in a continuous conductive track, and where the retraction movement of the needle guard generates the electrical state change.

In a seventh aspect of the invention, the present disclosure encompasses a method of detecting an extension movement of a needle guard within an autoinjector including sensing an electrical state change as the needle guard extends when an insertion force is removed after inserting a needle into a user, where the needle guard includes a region configured to generate one or more electrical breaks in a continuous conductive track, and where the extension movement of the needle guard generates the electrical state change.

According to the fifth, sixth or seventh aspect of the invention, the autoinjector includes an audible clicker configured to produce one or more audible clicks during a dispensing movement of a plunger rod of the autoinjector. Optionally, the audible clicker may include a ring surrounding the plunger rod, and where the ring includes a deflectable protrusion configured to contact the plunger rod and produce the one or more audible clicks. Further optionally, the plunger rod may include a ridged surface configured to contact the deflectable protrusion of the audible clicker. Each ridge of the ridged surface may be configured to cause the deflectable protrusion to deflect during the dispensing movement of the plunger rod.

According to the fifth, sixth or seventh aspect of the invention, a first sensor is disposed between a proximal end and a distal end of the housing, and the first sensor is configured to detect the electrical state change generated by the dispensing movement, the retraction movement, and/or the extension movement. Optionally, the first sensor may include a Near Field Communication (NFC) chip, an NFC coil, or an NFC tag. Additionally or alternatively, the first sensor may be further configured to detect an ambient temperature in proximity to a container.

The fifth, sixth or seventh aspect of the invention, additionally or alternatively, may include sending, via a Near Field Communication module, signals from a first sensor, if present, to a mobile device. Additionally, the fifth, sixth or seventh aspect of the invention may include displaying information about the dispensing movement on the mobile device.

The fifth, sixth or seventh aspect of the invention may include measuring a temperature of medicament within the autoinjector or an ambient temperature within the autoinjector using a temperature sensor. Optionally, the fifth, sixth or seventh aspect of the invention may include sending, via a Near Field Communication module, signals from the temperature sensor to a mobile device. Additionally or alternatively, the fifth, sixth or seventh aspect of the invention may include displaying the temperature of the medicament or the ambient temperature on the mobile device; indicating that the temperature of the medicament or the ambient temperature is above a threshold temperature for use of the autoinjector; or both.

According to an eighth aspect of the invention, also provided is a composition comprising lenacapavir or a pharmaceutically accepted salt thereof for use in the prevention or treatment of HIV, where the composition is administered via an autoinjector (e.g., any described herein, such as those of the first, second or third aspects of the invention). The administration may be subcutaneous or intramuscular.

According to a ninth aspect of the invention, also provided is the use of lenacapavir or a pharmaceutically accepted salt thereof for the manufacture of a medicament for the prevention or treatment of HIV, wherein the prevention or treatment comprises administering the medicament via an autoinjector (e.g., any described herein, such as those of the first, second or third aspects of the invention). The administration may be subcutaneous or intramuscular.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIG. 1 illustrates an example of an autoinjector.

FIG. 2 is a cut-away view of an example autoinjector.

FIG. 3 is a cross section view of another example autoinjector.

FIG. 4A illustrates example conductive tracks of an autoinjector.

FIG. 4B illustrates an example plunger rod of an autoinjector.

FIG. 4C illustrates an example needle guard of an autoinjector.

FIG. 5A illustrates a top view of an example linkage of an autoinjector.

FIG. 5B illustrates a bottom view of the example linkage of FIG. 5A.

FIG. 6A-6F illustrates an example autoinjector during operation of the autoinjector.

FIG. 7 is an example electronics design for an autoinjector.

FIG. 8 is a flow chart for an example method of dispensing a dose using an autoinjector.

FIG. 9 is an example plot of temperature readings of a medicament using an example autoinjector.

Like reference numbers and designations in the various drawings indicate like elements.

### DETAILED DESCRIPTION

The present systems, devices, and methods can sense a dispensing movement of a plunger rod within an autoinjector. For example, one or more continuous conductive tracks can be configured to be cut during the dispensing movement, which in turn generates an electrical state change that can be sensed by a sensing assembly to track the dispensing movement of the plunger rod. The electrical state change can be used to generate an electrical signal, which can include voltage measurements or other electrical measurements (e.g., resistance measurements and the like) that indicate that the plunger rod has reached a certain location within the autoinjector or has progressed to the end of the injection. The devices can include a sensing assembly that can determine that the plunger rod has completed the dispensing movement after receiving one or more electrical signals. If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth or inserted with sufficient insertion force. For example, a needle guard can be configured to retract when an insertion force is applied to the needle and configured to extend when the insertion force is removed, and an electrical state change can be generated for a retraction movement when sufficient insertion force is applied to needle and/or for an extension movement of the needle guard when the insertion force is removed. Determining that the needle is sufficiently inserted into the patient can be advantageous because if the needle is not sufficiently inserted into the patient, the patient may not receive the medicament properly.

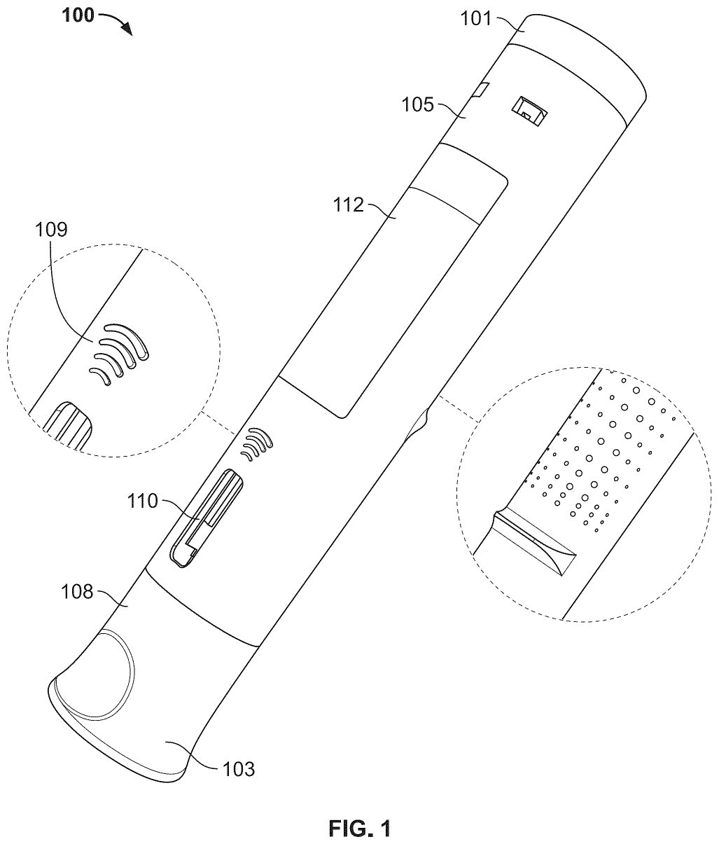

FIG. 1 illustrates an autoinjector 100 that can sense a dispensing movement of a plunger rod within the autoinjector 100 and sense whether a needle of the autoinjector 100 is inserted into a patient with sufficient insertion force. Alternatively, or in addition, the autoinjector 100 can sense whether an insertion force is removed during or after dispensing or delivery. The autoinjector 100 includes a housing 105, a proximal end 101 of the housing 105, and a front cap 108 attached to a distal end 103 of the housing 105. The front cap 108 covers a needle assembly configured to be inserted into the user during injection. The user removes the front cap 108 prior to use of the autoinjector 100. A sensing assembly can be located somewhere between the proximal end 101 and the distal end 103 of the housing 105. A sensing assembly may be located within a connectivity region 109. The connectivity region may be configured to transmit an electrical signal indicative of an electrical state change (e.g., as described herein) from a sensor to an external device (e.g., a mobile device, smartphone, or tablet) through a wireless communication protocol (e.g., near field communication, short range radio communication, wireless transfer protocol, Wi-Fi). The sensing assembly can sense a dispensing movement of a plunger rod within the autoinjector 100, e.g., to determine that the plunger rod has completed the dispensing movement, as discussed below. The housing 105 can include flanges and/or textured regions configured to accommodate the user's fingers.

The housing 105 can include an optional label 112 that provides information about the autoinjector 100. For example, the label 112 can include medicament information, such as the type of medicament, the size of the dose, and the delivery time of the dose. Optionally, the housing 105 does not include a label 112.

The housing 105 also includes an optional window 110, through which a user can see medicament contained within the autoinjector 100, e.g., within a container of the autoinjector 100 (see discussion below). The window 110 may help a user determine whether the autoinjector 100 has been used. Before use of the autoinjector 100, the user can see through the window 110 to determine whether there is medicament within the autoinjector 100, for example, to determine that the autoinjector has not been used. During use of the autoinjector 100, the user may look through the window 110 to determine whether the volume of medicament in the autoinjector 100 is decreasing. After use of the autoinjector 100, the user may look through the window 110 to determine that there is no medicament in the autoinjector 100, for example, to determine that the autoinjector 100 has been used.

Different injection sites, patient age and patient body mass may affect the recommended needle length, and higher viscosity drugs will require a larger diameter needle to prevent the injection force becoming too high for the device. Advantageously, selecting needle gauge based on viscosity of drug being administered can ensure that the full dose of drug is administered without undue strain. Typically, injection force is less than 40 Newtons through needle gauge selection. Preferably, injection force is less than 20 Newtons through needle gauge selection.

Needle gauges disclosed herein are provided in Birmingham Wire Gauge (also known as: Birmingham Gauge or Stubs Iron Wire Gauge), abbreviated as “gauge” or G. In accordance with ISO standard ISO 9626:2016, needle wall thickness designations include Regular Wall, Thin Wall, Extra Thin Wall, and Ultra Thin Wall. Regular Wall thickness is abbreviated to RW. Thin Wall thickness is abbreviated to TW. Extra Thin Wall thickness is abbreviated to ETW. Ultra Thin Wall is abbreviated to UTW. Alternatively, needle wall thickness may be Special Thin Wall; Special Thin Wall thickness is abbreviated as STW. Viscosity is provided in centipoise (cP), where one centipoise is equivalent to one millipascal-second.

The autoinjector 100 may be used for subcutaneous injections, which are directed into fat tissue between the skin and the muscle of the patient. Subcutaneous injections typically involve shorter and narrower needles than intramuscular injections, which are directed into the muscle of a patient. Needles for subcutaneous injections are typically 34-27 gauge and 4-12 mm in insertion depth (needle extension) for subcutaneous injections into the abdomen. Insertion depth for a subcutaneous injection may be 4-8 mm. For subcutaneous injection, needle length may be 8-13 mm. For delivery of 2.25-3 mL dose of liquid (medicament) with a viscosity of up to 5 cP using a needle with a needle length 8-13 mm, needle gauge may be 29 G RW or TW, or 27 G RW. For delivery of 2.25-3 mL dose of liquid (medicament) with a viscosity of up to 30 cP using a needle with a needle length 8-13 mm, needle gauge may be 27 G TW or 25 G RW. For delivery of 2.25-3 mL dose of liquid (medicament) with a viscosity of up to 50 cP using a needle with a needle length 8-13 mm, needle gauge may be 25 G TW. For delivery of 2.25-3 mL dose of liquid (medicament) with a viscosity of up to 160 cP using a needle with a needle length 8-13 mm, needle gauge may be 25 G STW or 23 G RW. For delivery of 2.25-3 mL dose of liquid (medicament) with a viscosity of up to 300 cP using a needle with a needle length 8-13 mm, needle gauge may be 22 G ETW. For delivery of 2.25-3 mL dose of liquid (medicament) with a viscosity of up to 600 cP using a needle with a needle length 8-13 mm, needle gauge may be 18 G ETW or 18 G UTW. For delivery of 1 mL dose of liquid (medicament) with a viscosity of up to 10 cP using a needle with a needle length 8-13 mm, needle gauge may be 29 G RW or TW, or 27 G RW. For delivery of 1 mL dose of liquid (medicament) with a viscosity of up to 30 cP using a needle with a needle length 8-13 mm, needle gauge may be 27 G TW or 25 G RW. For delivery of 1 mL dose of liquid (medicament) with a viscosity of up to 80 cP using a needle with a needle length 8-13 mm, needle gauge may be 25 G TW. For delivery of 1 mL dose of liquid (medicament) with a viscosity of up to 350 cP using a needle with a needle length 8-13 mm, needle gauge may be 25 G STW or 23 G RW. For delivery of 1 mL dose of liquid (medicament) with a viscosity of up to 600 cP using a needle with a needle length 8-13 mm, needle gauge may be 18-22 G ETW.

The autoinjector 100 may be used for intramuscular injections. Needles for intramuscular injections on adults are typically 25-20 gauge and 15-25 mm in insertion depth (needle extension). Alternatively, insertion depth for an intramuscular injection may be 25-50 mm. For intramuscular injection, needle length may be 1-1.5 inches (25.4-38.1 mm). For delivery of 2.25-3 mL dose of liquid (medicament) with a viscosity of up to 1 cP using a needle with a needle length 25.4-38.1 mm, needle gauge may be 29 G RW or TW, or 27 G RW. For delivery of 2.25-3 mL dose of liquid (medicament) with a viscosity of up to 5 cP using a needle with a needle length 25.4-38.1 mm, needle gauge may be 27 G TW or 25 G RW. For delivery of 2.25-3 mL dose of liquid (medicament) with a viscosity of up to 10 cP using a needle with a needle length 25.4-38.1 mm, needle gauge may be 25 G TW. For delivery of 2.25-3 mL dose of liquid (medicament) with a viscosity of up to 40 cP using a needle with a needle length 25.4-38.1 mm, needle gauge may be 25 G STW or 23 G RW. For delivery of 2.25-3 mL dose of liquid (medicament) with a viscosity of up to 200 cP using a needle with a needle length 25.4-38.1 mm, needle gauge may be 22 G ETW. For delivery of 2.25-3 mL dose of liquid (medicament) with a viscosity of up to 600 cP using a needle with a needle length 25.4-38.1 mm, needle gauge may be 18 G ETW or 18 G UTW. For delivery of 1 mL dose of liquid (medicament) with a viscosity of up to 5 cP using a needle with a needle length 25.4-38.1 mm, needle gauge may be 29 G RW or TW, or 27 G RW. For delivery of 1 mL dose of liquid (medicament) with a viscosity of up to 10 cP using a needle with a needle length 25.4-38.1 mm, needle gauge may be 27 G TW or 25 G RW. For delivery of 1 mL dose of liquid (medicament) with a viscosity of up to 30 cP using a needle with a needle length 25.4-38.1 mm, needle gauge may be 25 G TW. For delivery of 1 mL dose of liquid (medicament) with a viscosity of up to 50 cP using a needle with a needle length 25.4-38.1 mm, needle gauge may be 25 G STW or 23 G RW. For delivery of 1 mL dose of liquid (medicament) with a viscosity of greater than 50 cP using a needle with a needle length 25.4-38.1 mm, needle gauge may be 18-22 G ETW.

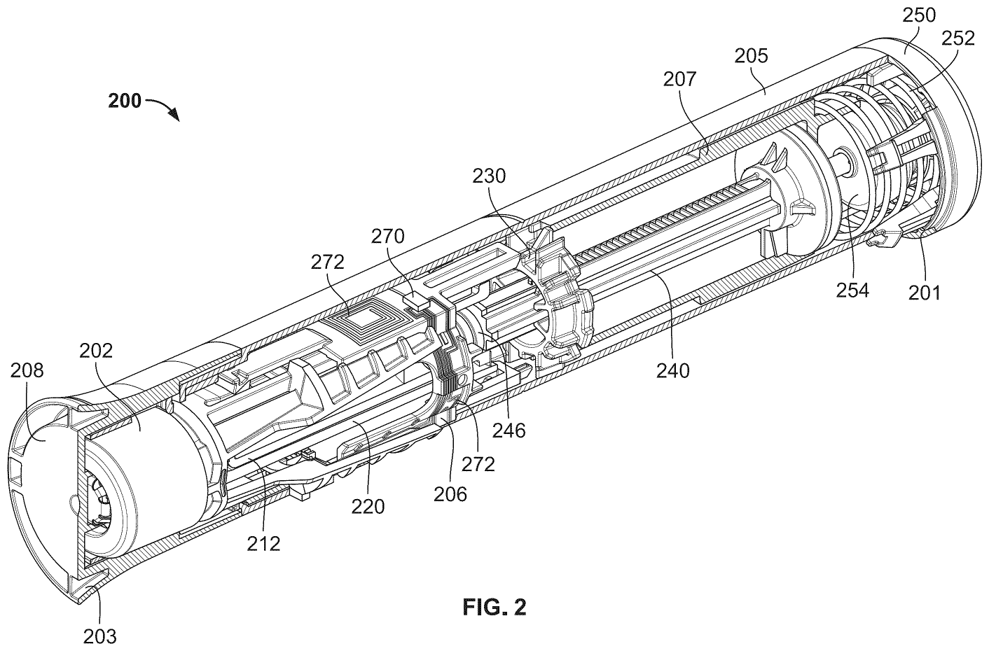

FIG. 2 is a cut-away view of an example autoinjector 200. The autoinjector 200 includes a housing 205 and a front cap 208. The front cap 208 is disposed at a distal end 203 of the housing 205. When installed on the autoinjector 200 (as shown in FIG. 2), the front cap 208 covers the needle guard 202. The user removes the front cap 208 from the autoinjector 200 prior to use of the autoinjector 200. The needle guard 202 protects the needle when the autoinjector 200 is not in use. The needle guard 202 is configured to retract when an insertion force is applied to the needle, thereby undergoing a retraction movement. Also, the needle guard 202 is configured to extend when the insertion force is removed, thereby undergoing an extension movement. By tracking the occurrence of such retraction and extension movements, dose start and dose end events can be detected (e.g., as described herein).

The autoinjector 200 includes a container 212 (e.g., a syringe) within the housing 205 for holding a medicament to be injected. The container 212 may have an internal volume of 1.5-3 mL. The container 212 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL. The container 212 is disposed in a syringe carrier 220. Optionally, a syringe backstop holds and maintains the position of the container 212 within the housing 205. A plunger 246 is slidably disposed within the container 212. A plunger rod 240 is configured to push the plunger 246 through the container 212 to dispense the medicament through the needle. The plunger rod 240 protrudes through a lock ring 230. The plunger rod 240 and lock ring 230 are disposed within a delivery chamber 207.

A transfer sleeve 206 is located within the housing 205 and between the needle guard 202 and the lock ring 230. Furthermore, an outer surface portion of the transfer sleeve 206 provides one or more conductive tracks 272. Movement of the needle guard 202 and/or the plunger rod 240 can result in cutting one or more conductive tracks 272, thereby forming one or more non-continuous conductive tracks. Non-continuous conductive track(s) can be sensed in any useful manner, such as by an electrical state change due to the lack of a conductive path because the conductive track is now broken. By sensing the presence of such non-continuous conductive track(s), movements of the needle guard 202 and/or the plunger rod 240 can be detected by one or more sensors. Such sensors may include an NFC chip 270 and/or an NFC coil 272. A sensing assembly may include one or more passive components. A sensing assembly may include one or more conductive tracks, NFC coils, NFC chips, or a combination of any of these.

At a proximal end 201 of the autoinjector 200, a rear case 250 is coupled to the housing 205. The proximal end 201 also includes a spring 252 (e.g., an anti-rattle spring) and a gas canister assembly 254. The spring 252 is disposed between the rear case 250 and the delivery chamber 207. The spring 252 biases the delivery chamber 207 toward the distal end 203. The gas canister assembly 254 includes pressurized gas that when released provides a force acting on the plunger rod 240 to push the plunger through the container 212 in a dispensing movement to dispense the medicament. During a dispensing movement, the pressurized gas is contained in the delivery chamber 207, and a piston seal can reduce leakage around the plunger rod 240.

Activation of the gas canister assembly 254 can occur by a user fully depressing the needle guard 202 into the housing 205 (e.g., by pressing the needle guard 202 and the autoinjector 200 against his or her skin with a sufficient insertion force), such that the needle guard 202 moves the transfer sleeve 206 proximally. In turn, this causes all of the internal components (e.g., including the delivery chamber 207) to move a relatively small proximal distance against the bias of the spring 252 disposed at a proximal end 201 of the autoinjector 200. For example, the relatively small proximal distance can be a distance of 1-10 millimeters. Upon moving such a relatively small proximal distance, the gas canister assembly 254 can be activated (e.g., by movement of a firing pin that pierces an end of a gas canister within the gas canister assembly 254), thereby releasing compressed gas from within. The compressed gas can include, for example, argon, carbon dioxide, krypton, xenon, etc. Depression of the needle guard 202 (e.g., to provide a retraction movement of the needle guard) can cut one or more conductive tracks 272. Optionally, depression of the needle guard 202 can cause an internal component to contact a sensing assembly including a force sensor, such that the force sensor can measure the insertion force of the needle into the user (e.g., as described herein).

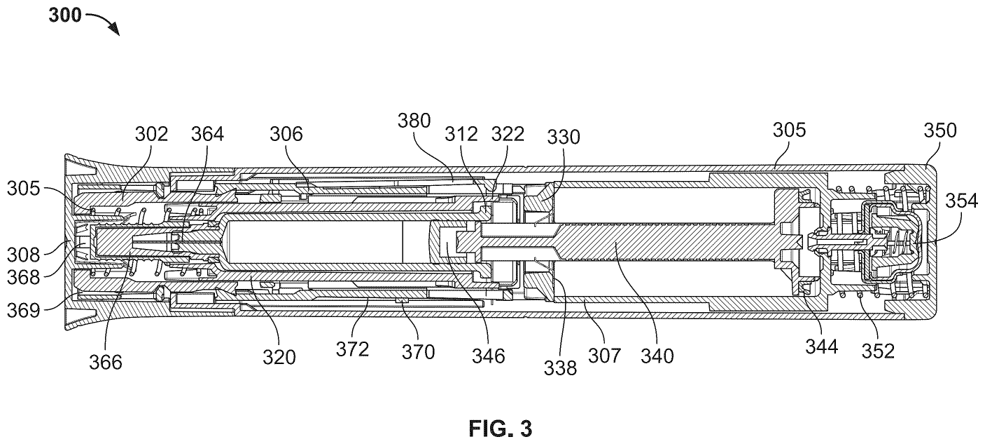

FIG. 3 is a cross-section view of yet another example autoinjector 300. The autoinjector 300 includes a housing 305 and front cap 308 coupled to a distal end of the housing 305. The front cap 308 includes a needle shield remover 368 that can remove a needle shield 366 from a needle 364 when the front cap 308 is removed from the housing 305. The front cap 308 also includes an anti-drop ring 369 to reduce the likelihood of accidental removal of the front cap 308 due to the autoinjector 300 being dropped by a user.

The needle 364 is connected to container 312. The needle shield 366 and needle guard 302 protect the needle 364 prior to use of the autoinjector 300. The needle guard 302 is biased away from the container 312 by the needle guard spring 305. A needle guard spring 305 is disposed between the needle guard 302 and the syringe carrier 320. The needle guard spring 309 biases the needle guard 302 toward the distal end of the housing 305, causing the needle guard 302 to extend from the housing 305 and cover the needle 364. Retraction of the needle guard 302 compresses the needle guard spring 309. The needle guard 302 can be retracted by exerting a force on the distal end (e.g., by applying an insertion force to the needle 364). Upon removing the sufficient force applied to the autoinjector 300 (e.g., after the dose is dispensed), the needle guard 302 can then extend from the housing to cover the needle 364 (e.g., in a retraction movement).

The container 312 can include a medicament to be injected into a patient. The container 312 may have an internal volume of 1.5-3 mL The container 312 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL The container 312 is held in place within the autoinjector 300 by syringe carrier 320. A syringe backstop 322 holds the syringe carrier 320 in place restricting movement of the syringe carrier 320 toward the rear case 350.

A transfer sleeve 306 is positioned around the syringe carrier 322. The transfer sleeve 306 interfaces between the needle guard 302 and the delivery chamber 307. The conductive track 372 and an NFC chip 370 can be located on the transfer sleeve 306. A linkage 380 is disposed within the housing 305 and configured to interact with the transfer sleeve 306. The linkage 380 may provide a structural latch between the needle guard 302 and the lock ring 330. Interactions between the needle guard 302, the linkage 380, the plunger 340, and the transfer sleeve 306 can be used to track a retraction movement, an extension movement, and a dispensing movement.

The plunger rod 340 protrudes through a lock ring 330 to exert a force on the plunger 346 that is slidably disposed within the container 312 to dispense the contents of the container 312. The autoinjector 300 can also include a circular clicker 338 (e.g., a ring configured to include an opening therein) disposed on a proximal side of the lock ring 330. The circular clicker 338 interfaces with the plunger rod 340. As can be seen, openings and other structural elements (e.g., contacts, stops, arms, etc.) for the lock ring 330 and the circular clicker 338 can be configured to minimize impeding a pathway for the dispensing movement of the plunger rod 340. As the plunger rod 340 moves through the circular clicker 338 during a dispensing movement, the circular clicker 338 emits one or more audible click sounds that can be used to track the dispensing movement. For example, the circular clicker 338 can have a deflectable protrusion configured to contact a surface of the plunger rod 340. The dispensing movement of the plunger rod 340 can deflect the deflectable protrusion to cause the circular clicker 338 to produce one or more audible clicks, vibrations or the like. A surface of the plunger rod 340 can be configured to deflect the deflectable protrusion of the circular clicker 338.

The circular clicker 338 can include multiple deflectable protrusions. Where the circular clicker 338 includes multiple deflectable protrusions, the plunger rod 340 can include multiple surfaces configured to deflect the deflectable protrusions. For example, and without limitation, the plunger rod 340 can include multiple surfaces configured to deflect the deflectable protrusions, in which each surface can in turn include a plurality of ridges, bumps, projections, or other non-continuous structures along the surface to provide a ridged surface or other non-continuous surface. Alternatively, the circular clicker 338 may include a single deflectable protrusion. Where the circular clicker 238 includes a single deflectable protrusion, the plunger rod 340 can include a single surface configured to deflect the deflectable protrusion. Any number of deflectable protrusions can be employed to produce one or more clicks (e.g., a single deflectable protrusion or multiple deflectable protrusions). In one non-limiting example, a single deflectable protrusion can be employed to reduce the possibility of interference between multiple audible clicks. In another non-limiting example, multiple deflectable protrusions can be employed to operate in sync to provide a more distinct click sound, in which interference can be minimized, e.g., by control of tolerancing and/or alignment.

The circular clicker can be a portion of the autoinjector housing. For example, the autoinjector housing can include a deflectable protrusion that contacts the plunger rod 340. The plunger rod 340 can include one or more deflectable protrusions, and the housing (or another internal component of the autoinjector) includes a surface configured to deflect the one or more deflectable protrusions.

The delivery chamber 307 is disposed between the lock ring 330 and the gas canister assembly 354. The delivery chamber 307 includes the plunger rod 340. The piston seal 344 forms a seal between the plunger rod 340 and sides of the delivery chamber 307 to reduce leakage of the pressurized gas from the gas canister assembly 354 past the plunger rod 340. The gas canister assembly 354 is disposed between the delivery chamber 307 and the rear case 750. A spring 352 is disposed around the gas canister assembly 354. The spring 352 biases the delivery chamber 307 toward the needle 364.

The sensing assembly of the autoinjector can include a combination of one or more conductive tracks, which can be cut (e.g., mechanically or otherwise disconnected) to provide a non-continuous conductive track having a differing electrical state than a continuous conductive track. The sensing assembly can also include one or more components (e.g., electronic components) to sense an electrical state (e.g., by use of a sensor configured to generate an electrical signal based on the electrical state, such as an NFC chip, a voltage sensor, a resistance sensor), to detect temperature (e.g., a thermistor to measure an ambient temperature in the area in proximity to the container that includes the medicament), to detect insertion force (e.g., a force sensor to measure an insertion force to insert a needle into a user), to process data, and/or to communicate with an external device (e.g., a mobile device, smartphone, or tablet) through a wireless communication protocol (e.g., near field communication, short range radio communication, wireless transfer protocol, Wi-Fi). The sensing assembly may include one or more passive components. The sensing assembly may not include a battery.

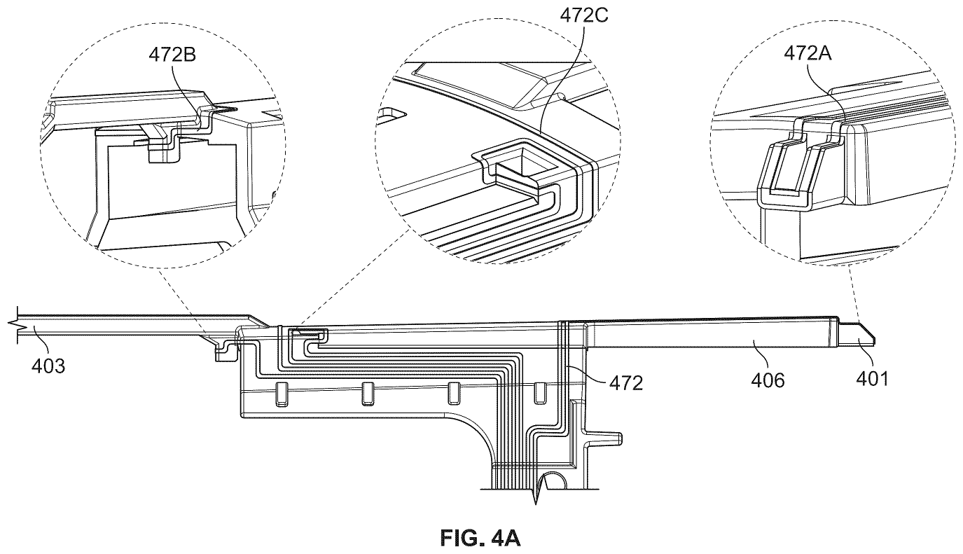

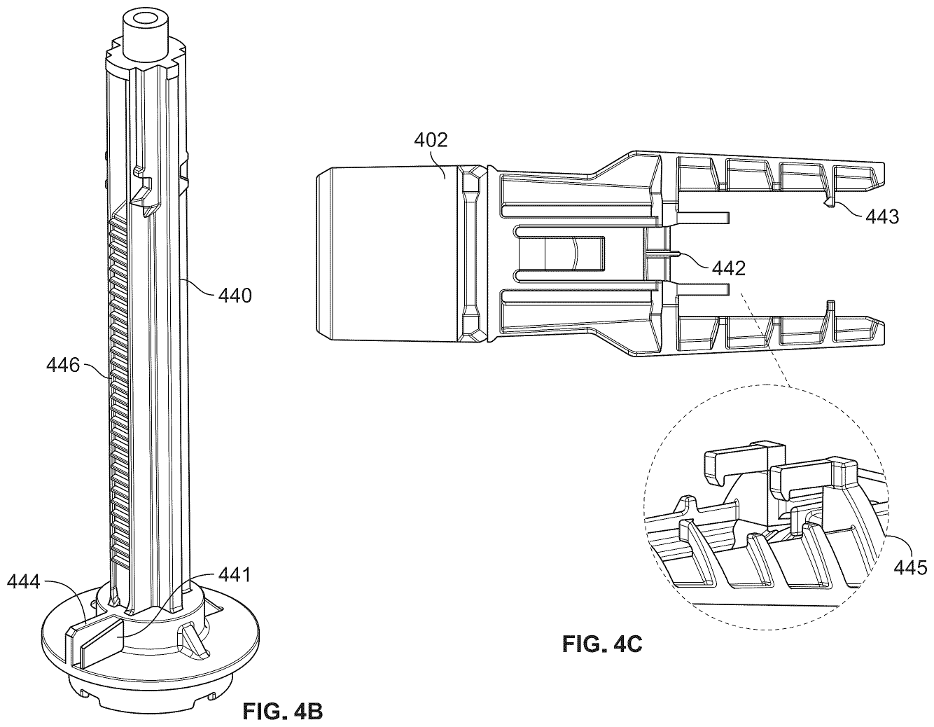

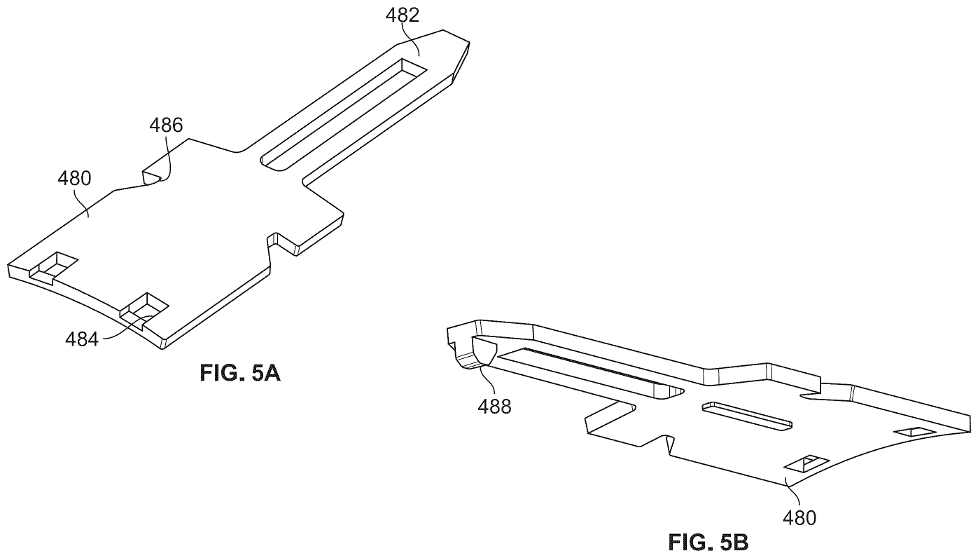

As seen in FIG. 4A, a transfer sleeve 406 can include one or more conductive tracks 472 disposed between the proximal end 401 and the distal end 403. The transfer sleeve 406 can also include an NFC antenna and tag. Conductive components (e.g., conductive tracks 472) can be disposed on the transfer sleeve 406 in any useful manner, such as laser discrete structuring (LDS), sintering, depositing, plating, coating, painting, spraying, printing, etching, patterning, annealing, or combinations thereof. The arrangement and configuration of the one or more conductive tracks 472 can allow for selective cutting based on certain interactions between the transfer sleeve 406 and other components of the autoinjector, such as the plunger rod 440 in FIG. 4B, the needle guard 402 in FIG. 4C, and/or the linkage 480 in FIGS. 5A-5B.

A first conductive track 472A may be disposed in proximity to the proximal end 401 of the transfer sleeve 406, in which an interaction between the first conductive track 472A and a proximal end of a plunger rod can provide cutting of the first conductive track 472A. FIG. 4B provides a non-limiting plunger rod 440, which includes a first cutter 441 disposed on the proximal end of the plunger rod 440. During operation, once the plunger rod 440 reaches the ends of its travel, the first cutter 441 acts to cut the first conductive track 472A, which can provide a first electrical state indicative of the end of the dosing. The dispensing movement of the plunger rod 440 may generate an electrical state change (e.g., a first electrical state change) arising from a non-continuous first conductive track. As such, the first electrical state change may be indicative of dose progression.

Optionally, the plunger rod 440 can include an unlatching feature 444, which unlatches the linkage 480 from the lock ring at the end of dosing. Also optionally, the plunger rod 440 can include a surface 446 having one or more ridges, in which a ridged surface can be configured to contact a deflectable protrusion (e.g., of an audible clicker, of a housing, or another appropriate component of the autoinjector). The ridged surface can include any appropriate number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of ridges. Each ridge of a ridged surface can deflect the deflectable protrusion to cause the deflectable protrusion to produce a signal (e.g., an audible click, a vibration, etc.) that can be sensed by a sensing assembly. For example, an example sensing output can include multiple signals, in which each signal corresponds to a ridge of a ridged surface. In turn, the number of signals can be used to track the number of ridges along the plunger rod 440 that passed by the circular clicker during a dispensing movement of the plunger rod 440. For example, a sensing assembly can use the received signals to determine that the plunger rod 440 has completed the dispensing movement after a predetermined number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of signals. The ridged surface can be any non-continuous surface configured to provide a plurality of non-continuous structures (e.g., bumps, projections, ribs, and the like upon that surface).

A second conductive track 472B may be disposed in proximity to the distal end 403 of the transfer sleeve 406, in which an interaction between the second conductive track 472B and a needle guard during a retraction movement can provide cutting of the second conductive track 472B. FIG. 4C provides a non-limiting needle guard 402, which includes a second cutter 442 disposed between the distal end and the proximal end of the needle guard 402. During operation, the second cutter 442 interacts with a distal end 403 of the transfer sleeve 406 to indicate a start of injection, e.g., such as by detecting a retraction movement of the needle guard when an injection force is applied to the needle. When the needle guard 402 retracts to expose the needle and then to interact with the transfer sleeve 406, the second cutter 442 cuts the second conductive track 472B. The retraction movement of the needle guard 402 may generate an electrical state change (e.g., a second electrical state change) arising from a non-continuous second conductive track.

A third conductive track 472C may disposed between the distal end 403 and proximal end of the transfer sleeve 406, in which an interaction between the third conductive track 472C and a needle guard during an extension movement can provide cutting of the third conductive track 472C. FIG. 4C provides a non-limiting needle guard 402, which includes a third cutter 443 disposed at the proximal end of the needle guard 402. During operation, the third cutter 443 interacts with a portion of the transfer sleeve 406 between the distal and proximal ends 403, 401 to indicate an end of injection, e.g., such as by detecting an extension movement of the needle guard when an injection force is removed from the needle. When the needle guard 402 extends to cover the needle, the third cutter 443 can cut the third conductive track 472C. Optionally, the needle guard 402 can include a needle guard latch 445, which creates a latch between the needle guard 402 and the linkage 480. The extension movement of the needle guard 402 may generate an electrical state change (e.g., a third electrical state change) arising from a non-continuous third conductive track.

FIG. 5A-5B provides a non-limiting linkage 480 including structural components to form one or more latches (e.g., reversible or irreversible latching or unlatching) between the linkage and the needle guard and/or the plunger rod. The linkage 480 can include a first latch feature 482 configured to create a latch between the linkage 480 and the lock ring (e.g., as described herein). This latch can optionally be unlatched by interaction between the unlatching feature 488 of the linkage 480 with the unlatching feature 444 on the plunger rod 440. Also included are features that create a non-returning latch between the linkage 480 and the needle guard 402. The linkage 480 can include a second latch feature 484 configured to create a latch between the linkage 480 and the needle guard 402. This latch can be initially latched and then later unlatched when the needle guard 402 undergoes a retraction movement. The linkage is still attached to the lock ring. Optionally, after unlatching, the linkage cannot be relatched. Optionally, the linkage can include one or more features to protect or expose one or more third conductive tracks in different states (e.g., by use of a guard feature 486 that may protect or expose a conductive track depending on the interaction of the linkage with the needle guard).

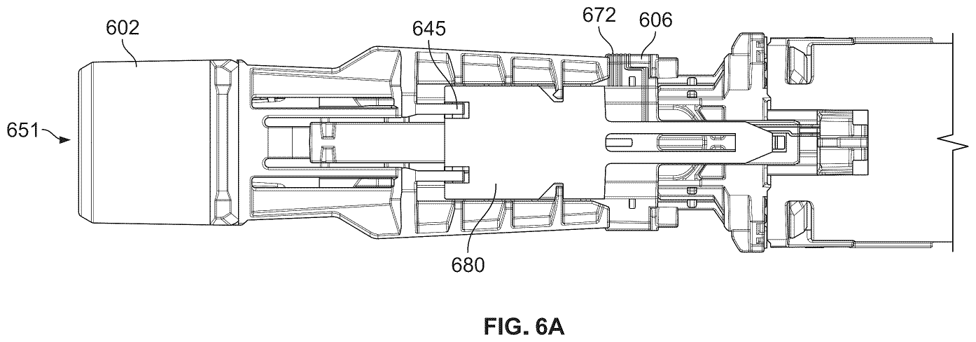

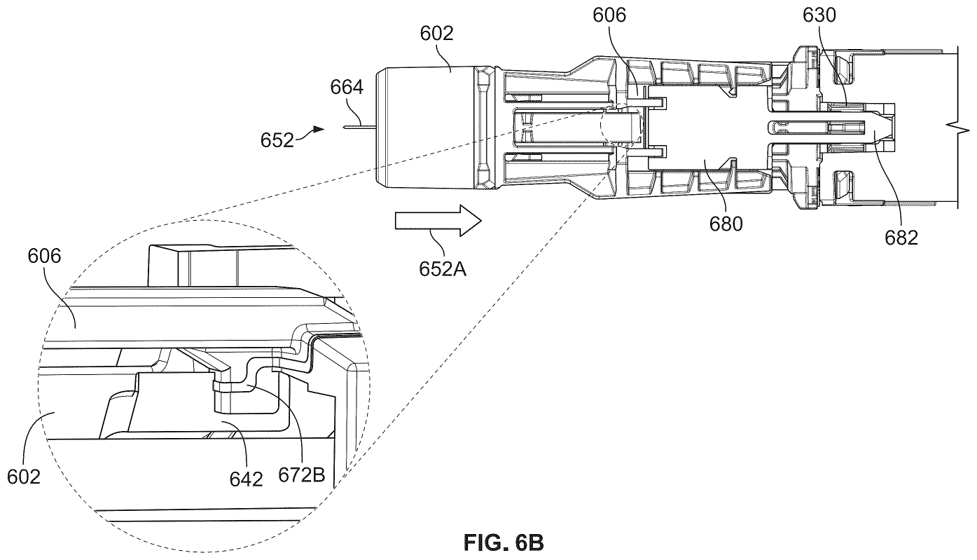

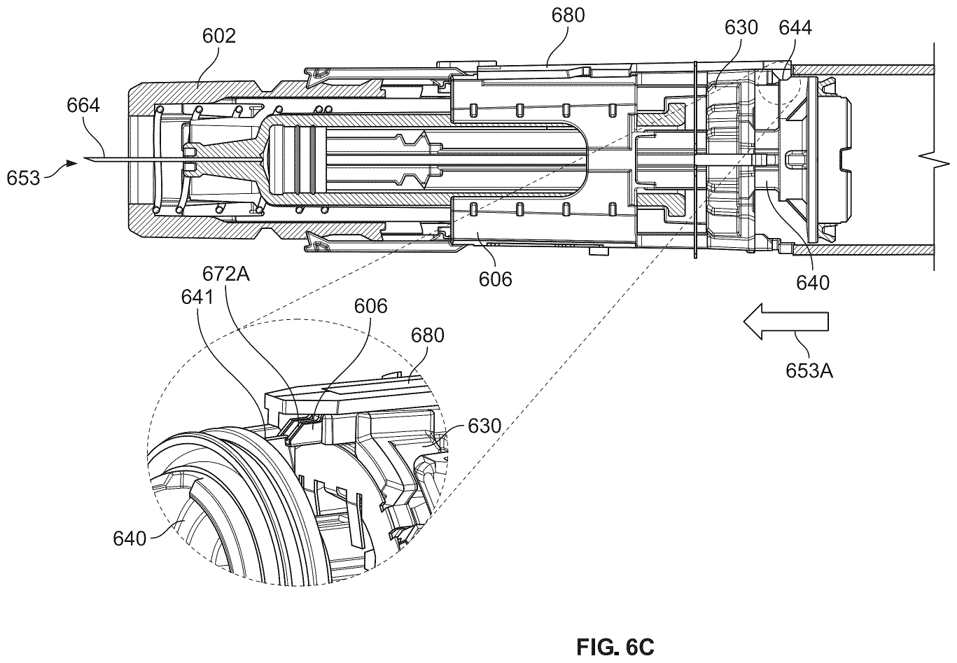

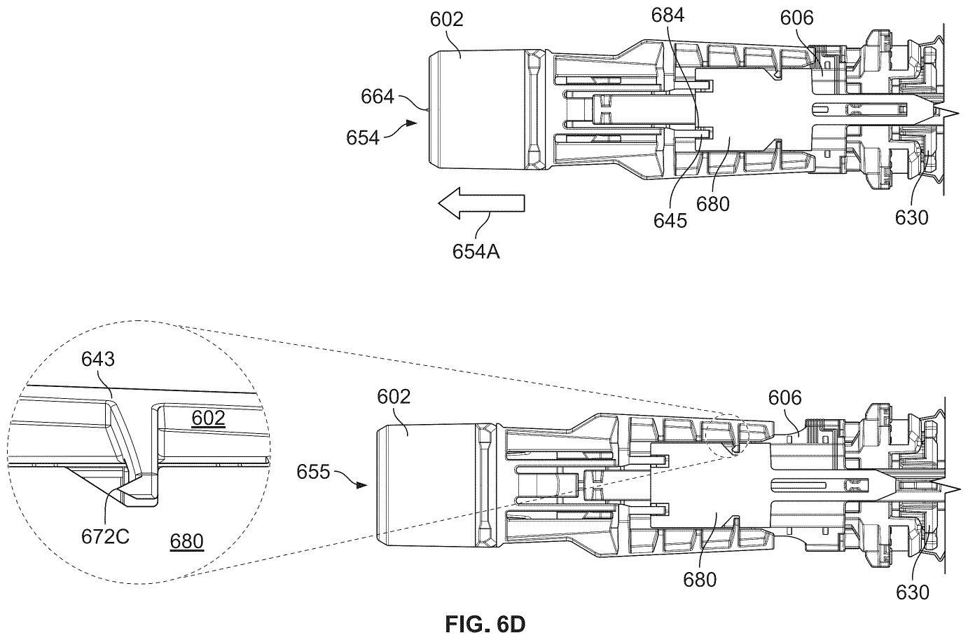

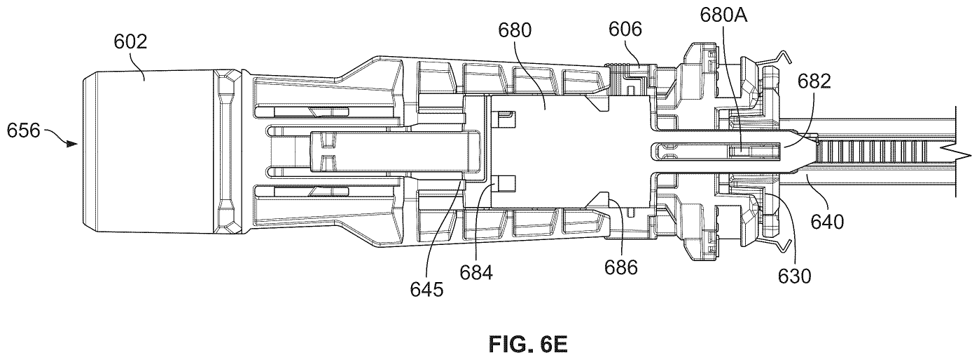

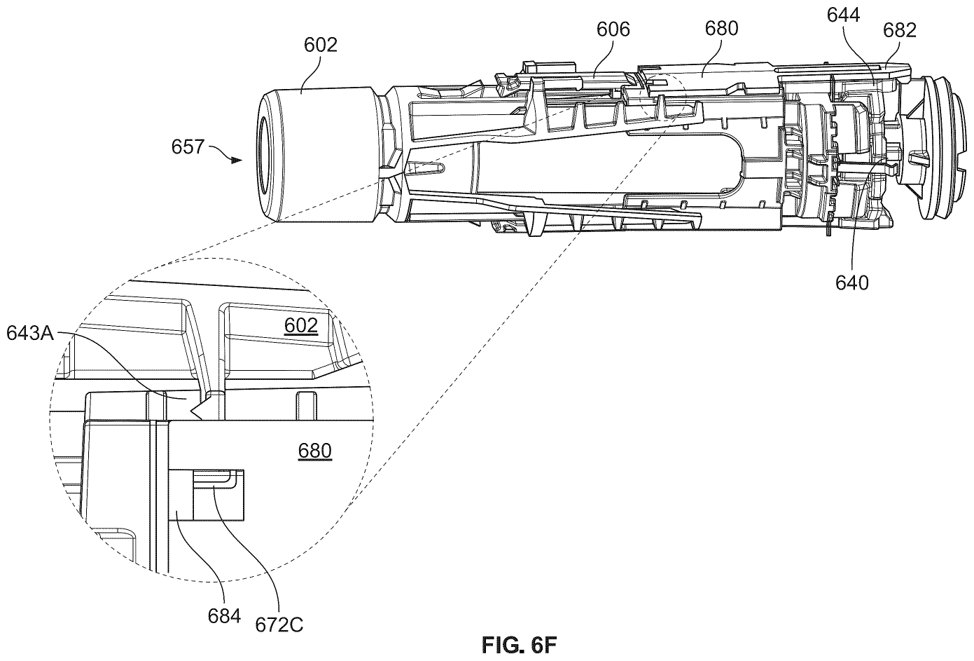

FIG. 6A-6F illustrates a dispensing movement, a retraction movement, and an extension movement of an example autoinjector. At position 651, the autoinjector is not yet used. The linkage 680 is latched to the needle guard 602 by way of the needle guard latch 645. All three conductive tracks 672 on the transfer sleeve 606 are intact (e.g., continuous), which is indicative of an unused device. At position 652, a force is exerted against the distal end of the needle guard 602 (e.g., by a user compressing the autoinjector against the skin) to provide a retraction movement 652A, and the needle 664 is now exposed though the needle guard 602. As retraction completes, the second cutter 642 on the needle guard 602 cuts the second conductive track 672B on the transfer sleeve 606, indicating the start of the injection. The linkage 680 is now latched to both the needle guard 602 (by way of the needle guard latch 645) and the lock ring 630 (by way of a first latch feature 682). If this track is the only track that is cut, then this is indicative of an incomplete dose (e.g., indicative of dose start but not dose end, such as from, e.g., blockage of the device, user error, etc.).

The movement of the needle guard 602 can activate the gas canister assembly to begin releasing pressurized gas into the delivery chamber. The lock ring 630 and plunger rod 640 translate toward the distal end of the autoinjector during a dispensing movement 653A to deliver the medicament. The plunger rod 640 can slide through the lock ring 630. The plunger rod 640 can include ridges that can interface with an audible clicker (e.g., by way of a circular clicker in proximity to the lock ring, as described herein) to generate audible clicks as the dose progresses. The audible clicks form an alternate way (or an additional way with the electrical state changes from the conductive tracks) of tracking dose progress during the dispensing movement.

At position 653, the dose is delivered, and the plunger rod 640 reaches the end of its travel. The first cutter 641 on the plunger rod 640 cuts the first conductive track 672A on proximal end of the transfer sleeve 606, giving an indication of the dose end. The linkage 680 is now unlatched from the lock ring 630 by an unlatching feature 644 on the proximal end of the plunger rod 640.

At position 654, the user removes the autoinjector from the skin, and the needle guard 602 undergoes an extension movement 654A to cover the needle 664. At position 655, the needle guard 602 completely covers the needle. As the linkage 680 is attached to the needle guard 602, the linkage also extends, thereby exposing the third conductive track 672C on the transfer sleeve 606. The third cutter 643 on the needle guard 602 then cuts the third conductive track 672C, indicating an end of the injection. When states related to cutting of the third conductive track (end of injection) is combined with that related to cutting of the first conductive track (start of dose) and cutting of the second conductive track (end of dose), this combination of electrical state changes is indicative of a successful dose.

Unsuccessful or failed states may be indicated based on particular combination of electrical state changes. At position 656, the user removes the autoinjector from the skin before the end of dose, and the linkage 680 remains latched to the lock ring 630 by way of an attached linkage portion 680A and the first latch feature 682. However, the linkage 680 unlatches from the needle guard 602 because of the spring driven motion of the needle guard 602. Thus, the third conductive track 672C remains protected (by the guard feature 686, which exposes the third conductive track 672C when the needle guard latch 645 of the needle guard 602 is attached to the second latch feature 684 of the linkage 680) and is not cut. When no tracks are cut, then this combination of electrical state changes is indicative of a failed state.

At position 657, the user prematurely lifts the autoinjector from the skin, but the dose continues to be delivered. When the plunger rod 640 reaches the end of its travel, the first cutter on the plunger rod 640 cuts the first conductive track 672A on the proximal end of the transfer sleeve 606. Then, the linkage 680 is unlatched from the lock ring 630 by an unlatching feature 644 on the plunger rod 44. The linkage 680 is also not latched to the needle guard 602, such that the blocked third cutter 643A cannot access the third conductive track 672C. When states related to cutting of the first conductive track (start of dose) is present but not the states related to the third conductive track (end of injection), this combination of electrical state changes is indicative of a premature lift-off.

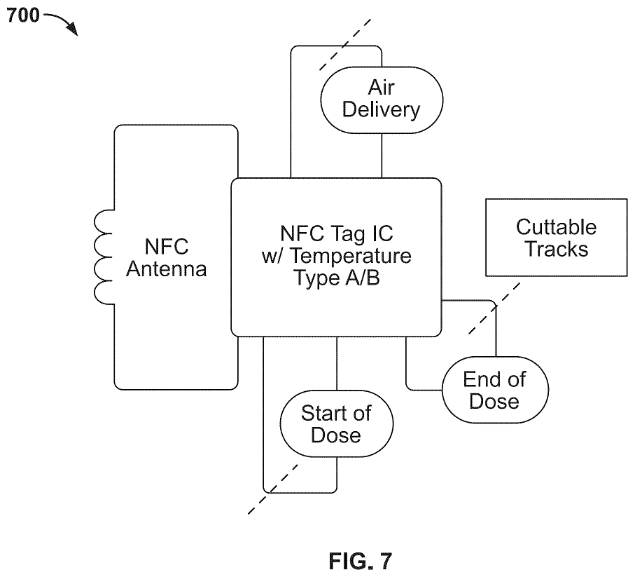

One or more electrical state changes of the conductive tracks can be maintained in the autoinjector, and such electrical state changes may later be detected by a sensor. For example, and without limitation, such state changes can be converted into electrical signals indicative of the electrical state change, e.g., by use of a sensor. Then, a connectivity region of the autoinjector can be configured to transmit an electrical signal indicative of said electrical state change from the sensor to an external device. FIG. 7 provides a non-limiting electronics design including an NFC tag integrated circuit (IC), an NFC antenna, and three cuttable conductive tracks. Additional NFC tag IC, NFC antenna, and conductive track may be present. An NFC chip may include the sensing assembly, which in turn can include temperature sensing and electrical state change sensing. Power may be provided to the sensing assembly by an external device (e.g., by tapping with a mobile phone).

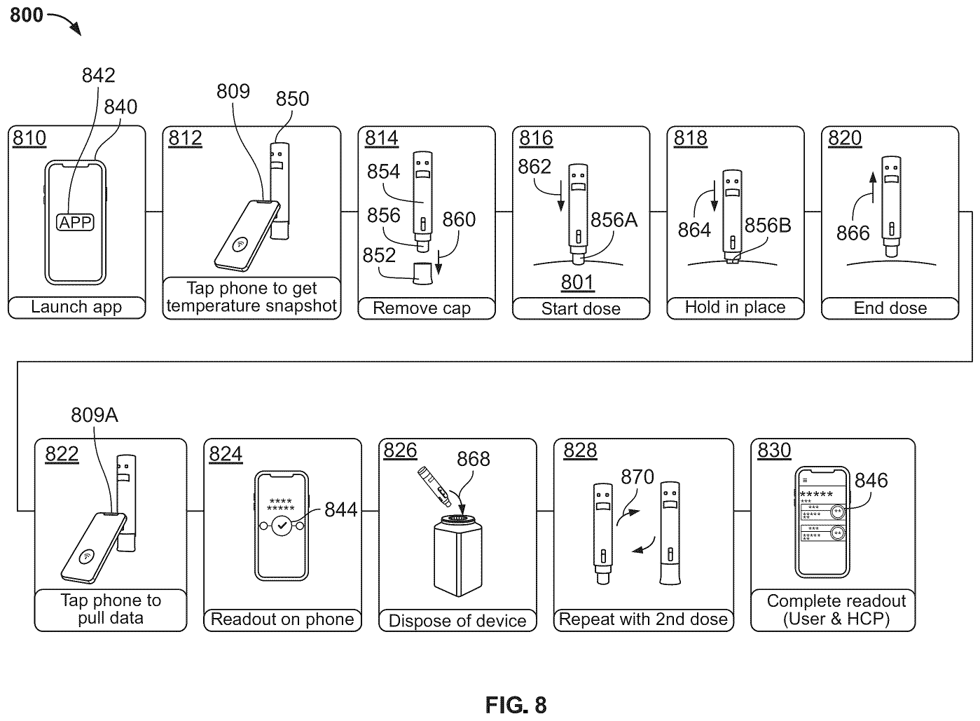

FIG. 8 illustrates an example method 800 of using an autoinjector 850. The autoinjector can be similar to, e.g., the autoinjectors 100, 200, 300, or any described herein. In the method 800, the autoinjector 850 is in communication with a mobile device 840. For example, the autoinjector can be in communication with the mobile device 840 via a near field communication protocol or wireless transfer protocol module (see discussion herein). In step 810, the method 800 includes launching an app 842 and/or website on the mobile device 840. In step 812, the method 800 includes tapping the connectivity region 809 on the housing of the autoinjector to wake up the electronics in the autoinjector 850. The method may include pressing a button to wake up the electronics in the autoinjector 850. For example, such a button can be included on the housing of the autoinjector 850. The button can be included on the app 1442 and/or website on the mobile device 1440. The button may not be activated, but, optionally, activation of a temperature sensor or a force sensor or electrical contact between elements in the autoinjector can wake up the electronics in the autoinjector. The electronics in the autoinjector can include, e.g., a sensing assembly, a continuous conductive track, a non-continuous conductive track, a voltage sensor, a resistance sensor, a temperature sensor (e.g., a thermistor), a force sensor (e.g., a force sensitive resistor, a load cell, a strain gauge, a force sense capacitor), a vibration sensor (e.g., an accelerometer, a microphone (e.g., a contact microphone or an air microphone), a displacement sensor, a velocity sensor)), a switch, and the like. Optionally, the voltage/resistance sensor can sense an electrical state change of a conductive track, as discussed above; the temperature sensor can sense a temperature in proximity to the medicament; the force sensor can sense an insertion force used to insert the needle of the autoinjector a sufficient distance into the patient; and/or the vibration sensor can sense a dispensing movement of a plunger rod that contacts an audible clicker, as discussed above.

In step 812, when turned on, the temperature sensor measures a temperature, and the near field communication protocol module can communicate the temperature to the mobile device 840. The temperature sensor can provide any number and type of measurements, including a temperature reading or a rate of change of temperature (e.g., 0.01-0.2° C./minute, 0.01-0.1° C./minute, 0.02-0.2° C./minute, 0.02-0.1° C./minute, about 0.05° C./minute, etc.) determined by readings determined over a time interval (e.g., a time interval of one, two, three, four, or five minutes, in which measurements can be taken every 10, 20, 30, 45, 60, or more seconds). The temperature may be an ambient temperature determined within the autoinjector. While not a direct measurement, the ambient temperature can be used to infer the temperature of the medicament. The temperature may be of a medicament contained within the autoinjector. The user can wait until the medicament is at an appropriate temperature for injection. For example, in many cases, users keep autoinjectors in a refrigerator or other chilled location. After the autoinjector is removed from the refrigerator, the medicament warms up to an appropriate temperature for injection (e.g., 5-10° C., 5-20° C., 5-30° C., 10-20° C., 10-30° C., 20-30° C., less than 30° C., etc.).

When the medicament is at an appropriate temperature, the autoinjector 850 is ready for use. In step 814, the user can remove 860 a front cap 852 of the ready autoinjector 854. Removing the cap 852 of the autoinjector 854 reveals the needle guard 856 so that the needle can be inserted into the patient (see discussion below). In step 816, the user brings 862 the needle guard 856 into contact with his or her skin 801 to start the dose. The needle guard 856 is initially in an extended position 856A where the needle is covered. At step 818, the user inserts 864 the needle by pressing the autoinjector further into the skin. Inserting the needle into the patient's skin moves the needle guard 856 into a retracted position 856B. A time delay may be present between a time for inserting the needle into the skin and a time for beginning the dose of medicament. The internal components of the autoinjector inject the medicament into the user when the user inserts the needle into his or her skin (see discussion below). The user holds the autoinjector in place during the injection. In step 820, the dose ends after the medicament has been injected into the user and the autoinjector is removed 866 from the patient.

In step 822, after the dose ends, the method 800 includes tapping the connectivity region 809A on the housing of the autoinjector to obtain information about the dose. In step 824, the method 800 includes displaying information 844 about the dose on the mobile device. For example, information can include whether the dose was successfully injected, the time of the delivery, the amount of medicament delivered, a likely error in delivery (e.g., early lift off, shallow delivery, etc.). The information can be determined based on received signals from, e.g., the force sensor, the temperature sensor, the vibration sensor, the mobile device.

In step 826, the user can dispose 868 of the autoinjector. The user can dispose of the entire autoinjector. Optionally, the user can remove the electronics from the autoinjector. In some non-limiting examples, the removed electronics can be disposed (e.g., to an appropriate waste stream). In other non-limiting examples, the removed electronics can be returned to the manufacturer for possible reassignment in a controlled manner. In step 828, the user can perform an injection with a second autoinjector by performing 870 the portions of the method 800 described above.

In step 830, the mobile device and/or another device, e.g., a device of a healthcare provider, can provide a full readout of information 846 from the one or more injections performed by the user. For example, the full readout of information can include information about each of the injections performed by the user, including whether each dose was successfully injected, the time of the delivery, the amount of medicament delivered, etc.

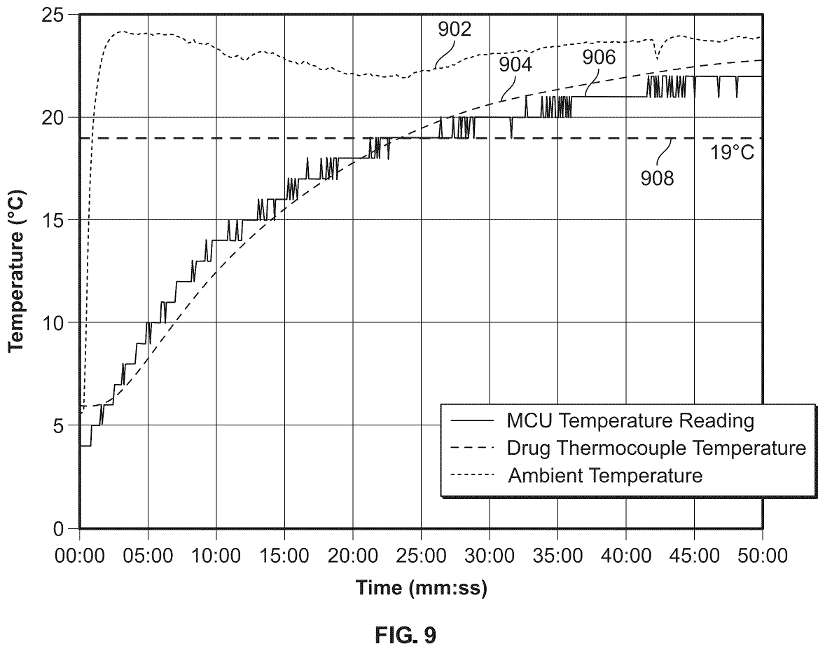

FIG. 9 is plot of temperature versus time. Three temperature traces are shown, ambient temperature 902, temperature of a drug 904 as measured by a thermocouple inserted into the drug, and a temperature reading 906 from a micro-control unit (MCU) with a thermistor. A threshold injection temperature 908 of 19° C. is also shown. When the drug is removed from a cooled location (e.g., a refrigerator), the ambient temperature quickly rises to normal room temperature. The MCU temperature reading 906 tracks the temperature of the drug 904 reasonably well indicating that the MCU temperature reading 906 is an adequate proxy for the temperature of the drug 904.

A thermistor (e.g., within the housing) can be used to measure (indirectly or directly) the temperature (e.g., an approximate temperature) of a medicament included in the container in the syringe carrier. Direct temperature of the medicament may impact sterility of the medicament. Indirect temperature reading of the medicament (e.g., by detecting an ambient temperature within the autoinjector and/or in proximity to the medicament) may be sufficient for dose initiation. A reading from the thermistor can be used to determine that the medicament is at a specified temperature to be injected into a patient. For example, the thermistor can measure the ambient temperature in the region near the syringe carrier and the temperature of the medicament can be inferred based on the ambient temperature near the syringe carrier.

As used herein, the term “medicament” refers to a pharmaceutical formulation containing at least one active pharmaceutical ingredient (API) which is formulated for administration via injection (e.g., a liquid formulation). The API may be a compound which acts as an anti-viral or has anti-viral properties. The anti-viral API may be a compound that can treat or prevent viral disease such as HIV infection, hepatitis, or other viral disease. The anti-viral API of the medicament may treat or prevent HIV infection alone or in combination with other drugs.

The API may be an anti-HIV drug such as a capsid inhibitor (e.g., an antiretroviral drug that targets the capsid protein shell of viruses). Examples of suitable capsid inhibitors can be found, for example, in U.S. Pat. Nos. 9,951,043; 10,071,985; 11,944,611; 11,787,825; and 12,084,467; and U.S. Publication No.: 20230212148.

The capsid inhibitor may be lenacapavir sodium (SUNLENCA). As used herein, “lenacapavir” (or LEN) refers to N-((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl) pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide.

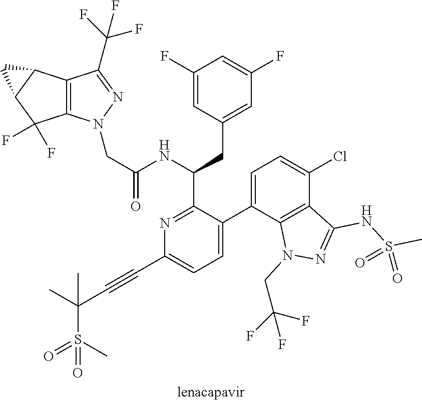 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Synthesis and characterization of lenacapavir, and salts thereof, are described, for example, in US patent publications US 20180051005 and US 20190300505. Various forms and/or uses of lenacapavir are disclosed, for example, in US 20190083478, US 20190084963, US 20200038389A1, and US 20210188815.

The capsid inhibitor may be lenacapavir, or a pharmaceutically acceptable salt thereof. The capsid inhibitor may be lenacapavir sodium. The capsid inhibitor may be lenacapavir (i.e., the free acid form of lenacapavir).

The present disclosure further includes medicaments (i.e., pharmaceutical compositions) comprising a capsid inhibitor provided herein, or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Pharmaceutical compositions of the disclosure are formulated so as to allow the active ingredients contained therein to be bioavailable upon administration of the composition to a patient. Compositions that will be administered to a patient take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the API, or a pharmaceutically acceptable salt thereof (e.g., for treatment or prevention of an HIV infection or reducing the risk of acquiring HIV).

Examples of suitable excipients are well known to the person skilled in the art of parenteral formulations and can be found, for example, in the Handbook of Pharmaceutical Excipients (eds. Rowe, Sheskey & Quinn), 6th edition 2009.

Examples of excipients in a parenteral formulation (for example, a subcutaneous or intramuscular formulation) include polyethylene glycol. In general, polyethylene glycol (PEG) is a polyether having a general formula H—(O—CH₂—CH₂)ₙ—OH. The PEG may be “capped” by an alkyl group. Optionally, the capped PEG may be of the formula alkyl-(O—CH₂—CH₂)ₙ—O-alkyl (for example, CH₃—(O—CH₂—CH₂)ₙ—OCH₃). The pharmaceutical compositions of the present disclosure can include PEG having an average molecular weight of about 100 to about 1000. The average molecular weight of PEG within the pharmaceutical composition may be about 100 to about 800. The average molecular weight of PEG within the pharmaceutical composition may be about 200 to about 600. The average molecular weight of PEG within the pharmaceutical composition may be about 400. The average molecular weight of PEG within the pharmaceutical composition may be about 300. The average molecular weight of PEG within the pharmaceutical composition may be about 200. In some embodiments of the pharmaceutical composition, different molecular weight PEG can be combined to obtain a desired property or properties (for example, viscosity). Specific examples of PEG include, but are not limited to, PEG 100, PEG 200, PEG 300, PEG 400, PEG 500, and PEG 600. PEG 100, for example, refers to a polyethylene glycol with an average molecular weight of about 100.

The pharmaceutical compositions of the present disclosure can be in the form of a sterile injectable preparation, such as a solution or sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution, and isotonic sodium chloride solution. In addition, sterile fixed oils can conventionally be employed as a solvent or suspending medium. For this purpose, any bland fixed oil can be employed, including synthetic mono- or diglycerides. In addition, fatty acids such as oleic acid can likewise be used in the preparation of injectables.

Example formulations suitable for use with the systems and devices of the present application can be found, for example, in U.S. Pat. No. 11,807,625.

The pharmaceutical composition may comprise the API (e.g., lenacapavir or lenacapavir sodium) at a concentration of about 10 mg/mL to about 600 mg/mL. The pharmaceutical composition may comprise the API (e.g., lenacapavir or lenacapavir sodium) at a concentration of about 100 mg/mL to about 400 mg/mL. The pharmaceutical composition may comprise the API (e.g., lenacapavir or lenacapavir sodium) at a concentration of about 250 mg/mL to about 350 mg/mL. The pharmaceutical composition may comprise the API (e.g., lenacapavir or lenacapavir sodium) at a concentration of about 300 mg/mL to about 325 mg/mL. The pharmaceutical composition may comprise the API (e.g., lenacapavir or lenacapavir sodium) at a concentration of about 305 mg/mL to about 310 mg/mL. The pharmaceutical composition may comprise the API (e.g., lenacapavir or lenacapavir sodium) at a concentration of about 309 mg/mL. The solution provided herein may comprise lenacapavir, or a pharmaceutically acceptable salt thereof, PEG 300, and water. The solution may comprise lenacapavir sodium, PEG 300, and water. The solution may comprise lenacapavir, PEG 300, and water.

The amount of water in the solution comprising lenacapavir sodium, PEG 300, and water may be about 10 w/w % to about 40 w/w %. The amount of water in the solution comprising lenacapavir sodium, PEG 300, and water may be about 15 w/w % to about 35 w/w %. The amount of water in the solution comprising lenacapavir sodium, PEG 300, and water may be about 20 w/w % to about 30 w/w %. The amount of water in the solution comprising lenacapavir sodium, PEG 300, and water may be about 21 w/w % to about 29 w/w %.

The amount of water in the solution comprising lenacapavir sodium, PEG 300, and water may be about 21.1 w/w % to about 27.5 w/w %. The amount of water in the solution comprising lenacapavir sodium, PEG 300, and water may be about 21.13 w/w % to about 27.47 w/w %. The amount of water in the solution comprising lenacapavir sodium, PEG 300, and water may be about 21.1 w/w %. The amount of water in the solution comprising lenacapavir sodium, PEG 300, and water may be about 21.13 w/w %. The amount of water in the solution comprising lenacapavir sodium, PEG 300, and water may be about 23 w/w %. The amount of water in the solution comprising lenacapavir sodium, PEG 300, and water may be about 23.4 w/w %. The amount of water in the solution comprising lenacapavir sodium, PEG 300, and water may be about 23.41 w/w %.

The amount of PEG 300 in the solution comprising lenacapavir sodium, PEG 300, and water may be about 35 w/w % to about 75 w/w %. The amount of PEG 300 in the solution comprising lenacapavir sodium, PEG 300, and water may be about 40 w/w % to about 55 w/w %. The amount of PEG 300 in the solution comprising lenacapavir sodium, PEG 300, and water may be about 43 w/w % to about 47 w/w %. The amount of PEG 300 in the solution comprising lenacapavir sodium, PEG 300, and water may be about 45 w/w %. The amount of PEG 300 in the solution comprising lenacapavir sodium, PEG 300, and water may be about 45.25 w/w %. The amount of PEG 300 in the solution comprising lenacapavir sodium, PEG 300, and water may be about 48 w/w % to about 52 w/w %. The amount of PEG 300 in the solution comprising lenacapavir sodium, PEG 300, and water may be about 50 w/w %. The amount of PEG 300 in the solution comprising lenacapavir sodium, PEG 300, and water may be about 50.1 w/w %. The amount of PEG 300 in the solution comprising lenacapavir sodium, PEG 300, and water may be about 50.13 w/w %.

The amount of lenacapavir sodium in the solution comprising PEG 300 and water may be about 5 w/w % to about 35 w/w %. The amount of lenacapavir sodium in the solution comprising PEG 300 and water may be about 20 w/w % to about 35 w/w %.

The amount of lenacapavir sodium in the solution comprising PEG 300 and water may be about 20 w/w % to about 30 w/w %. The amount of lenacapavir sodium in the solution comprising PEG 300 and water may be about 24 w/w % to about 28 w/w %. The amount of lenacapavir sodium in the solution comprising PEG 300 and water may be about 26.5 w/w %. The amount of lenacapavir sodium in the solution comprising PEG 300 and water may be about 26.46 w/w %.

The amount of lenacapavir sodium in the solution comprising PEG 300 and water may be about 30 w/w % to about 35 w/w %. The amount of lenacapavir sodium in the solution comprising PEG 300 and water may be about 32 w/w % to about 34 w/w %. The amount of lenacapavir sodium in the solution comprising PEG 300 and water may be about 33.6 w/w %. The amount of lenacapavir sodium in the solution comprising PEG 300 and water may be about 33.61 w/w %.

The solution may comprise about 10 w/w % to about 40 w/w % water, about 35 w/w % to about 75 w/w % PEG 300, and about 5 w/w % to about 45 w/w % of lenacapavir sodium. The solution may comprise about 10 w/w % to about 30 w/w % water, about 35 w/w % to about 65 w/w % PEG 300, and about 5 w/w % to about 45 w/w % of lenacapavir sodium.

The solution may comprise about 21.13 w/w % to about 27.47 w/w % water, about 45.25 w/w % to about 58.84 w/w % PEG 300, and about 13.69 w/w % to about 33.61 w/w % of lenacapavir sodium. The solution may comprise about 21.1 w/w % water, about 45.3 w/w % PEG 300, and about 33.6 w/w % of lenacapavir sodium. The solution may comprise about 21.13 w/w % water, about 45.25 w/w % PEG 300, and about 33.61 w/w % of lenacapavir sodium. The solution may comprise about 23.4 w/w % water, about 50.1 w/w % PEG 300, and about 26.5 w/w % of lenacapavir sodium. The solution may comprise about 23.41 w/w % water, about 50.13 w/w % PEG 300, and about 26.46 w/w % of lenacapavir sodium.

The solution provided herein may further comprise ethanol. The solution may comprise about 10 w/w % to about 40 w/w % water, about 20 w/w % to about 75 w/w % PEG 300, about 10 w/w % to about 70 w/w % of lenacapavir sodium, and about 1 w/w % to about 10 w/w % of ethanol. The solution may comprise about 10 w/w % to about 20 w/w % water, about 30 w/w % to about 40 w/w % PEG 300, about 37 w/w % to about 45 w/w % of lenacapavir sodium, and about 3 w/w % to about 8 w/w % of ethanol.

The solution may comprise about 16.93 w/w % water, about 36.22 w/w % PEG 300, about 41.85 w/w % of lenacapavir sodium, and about 5.00 w/w % ethanol. The solution may comprise about 16.9 w/w % water, about 36.2 w/w % PEG 300, about 41.9 w/w % of lenacapavir sodium, and about 5.0 w/w % ethanol.

The medicament which is administered by the injection device of the invention may be a long-acting injectable formulation which can be administered to a patient, for example, twice per month, once per month, once per quarter (e.g., every 3 months), twice per year (e.g., every 6 months), once per year, or less frequently.

Additionally, the medicament can include a formulation which has relatively high viscosity. The medicament may have a viscosity of 100-1000 cP. Preferably, the medicament may have a viscosity of 100-600 cP. For example, the medicament may have a viscosity of 250-500cP, 150-350 cP or 450-600 cP. The medicament may be a solution comprising lenacapavir sodium the medicament may have a viscosity of 250-500 cP, 150-350 cP or 450-600 cP.

The sensing assembly is intended to include various forms of digital computers, such as printed circuit boards (PCB), processors, digital circuitry, or otherwise parts of a system for determining dosing progression of an autoinjector. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The sensing assembly can include a processor, a memory, a storage device, and an input/output device (for example, sensors). Each of the components is interconnected using a system bus. The sensing assembly is capable of processing instructions for execution within the sensing assembly. The sensing assembly may be designed using any of a number of architectures. For example, the sensing assembly can include a CISC (Complex Instruction Set Computers) processor, a RISC (Reduced Instruction Set Computer) processor, or a MISC (Minimal Instruction Set Computer) processor.

In one implementation, the processor is a single-threaded processor. In another implementation, the processor is a multi-threaded processor. The processor is capable of processing instructions stored in the memory or on the storage device to display graphical information for a user interface on an input/output device.

The memory stores information within the sensing assembly. In one implementation, the memory is a computer-readable medium. In one implementation, the memory is a volatile memory unit. In another implementation, the memory is a non-volatile memory unit.

The storage device is capable of providing mass storage for the sensing assembly. In one implementation, the storage device is a computer-readable medium. In various different implementations, the storage device may be a floppy disk device, a hard disk device, an optical disk device, or a tape device.

The features described can be implemented in digital electronic circuitry, or in computer hardware, firmware, software, or in combinations of them. The features can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

A number of embodiments have been described. Nevertheless, it will be understood that various modifications may be made without departing from the spirit and scope of the disclosure. Accordingly, other embodiments are within the scope of the following claims.

### Embodiments

The following list provides embodiments of the invention and forms part of the description. These embodiments can be combined in any compatible combination beyond those expressly stated. The embodiments can also be combined with any compatible features described herein:

1. An autoinjector comprising:
a housing;
a connectivity region configured to be accessed at a region of the housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament;
a needle guard configured to retract when an insertion force is applied to the needle and configured to extend when the insertion force is removed;
wherein a dispensing movement of the plunger rod, a retraction movement of the needle guard, and/or an extension movement of the needle guard generates an electrical state change; and
a first sensor within the connectivity region configured to detect said electrical state change as the plunger rod and/or the needle guard moves.
   2. The autoinjector of embodiment 1, wherein the electrical state change is indicative of dose progression.
   3. The autoinjector of embodiment 1 or embodiment 2, wherein when the dispensing movement of the plunger rod generates the electrical state change, the electrical state change is configured to indicate completion of the dispensing movement of the plunger rod.
   4. The autoinjector of any of embodiments 1-3, wherein the container contains the medicament.
   5. The autoinjector of any preceding embodiment, wherein the medicament comprises lenacapavir or a pharmaceutically accepted salt thereof.
   6. The autoinjector of embodiment 5, wherein the medicament comprises lenacapavir sodium.
   7. The autoinjector of embodiment 6, wherein the medicament is a solution comprising: lenacapavir sodium, PEG 300 and water.
   8. The autoinjector of embodiment 7, wherein the solution comprises about 21.13 w/w % water, about 45.25 w/w % PEG 300, and about 33.61 w/w % of lenacapavir sodium.
   9. The autoinjector of embodiment 7, wherein the solution comprises about 23.41 w/w % water, about 50.13 w/w % PEG 300, and about 26.46 w/w % of lenacapavir sodium.
   10. The autoinjector of embodiment 7, wherein the solution further comprises ethanol, optionally wherein the solution comprises about 16.9 w/w % water, about 36.2 w/w % PEG 300, about 41.9 w/w % of lenacapavir sodium, and about 5.0 w/w % ethanol.
   11. The autoinjector of any preceding embodiment, wherein the autoinjector further comprises a gas canister assembly configured to release pressurized gas which, when released, provides a force acting on the plunger rod to push the plunger through the container.
   12. The autoinjector of any preceding embodiment, wherein the plunger rod comprises a first cutter region disposed on a proximal end of the plunger rod.
   13. The autoinjector of embodiment 12, wherein the first cutter region is configured to cut a first continuous conductive track during the dispensing movement of the plunger rod to provide the first non-continuous conductive track.
   14. The autoinjector of embodiment 13, wherein the first continuous conductive track is provided in proximity to the first cutter region of the plunger rod at an end of the dispensing movement.
   15. The autoinjector of any one of embodiments 0-14, wherein the needle guard is further configured to expose the needle when the insertion force is applied to insert the needle into a user and during the retraction movement of the needle guard, and wherein the retraction movement of the needle guard generates a second electrical state change arising from a second non-continuous conductive track.
   16. The autoinjector of embodiment 15, wherein the needle guard is further configured to cover the needle when the insertion force is removed to withdraw the needle from the user and during the extension movement of the needle guard, and wherein the extension movement of the needle guard generates a third electrical state change arising from a third non-continuous conductive track.
   17. The autoinjector of embodiment 15 or embodiment 16, wherein the needle guard comprises a second cutter region and/or a third cutter region.
   18. The autoinjector of embodiment 17, wherein the second cutter region is configured to cut a second continuous conductive track during the retraction movement of the needle guard to provide the second non-continuous conductive track and/or wherein the third cutter region is configured to cut a third continuous conductive track during the extension movement of the needle guard to provide the third non-continuous conductive track.
   19. The autoinjector of embodiment 18, wherein the second continuous conductive track is provided in proximity to the second cutter region of the needle guard at an end of the retraction movement and/or wherein the third continuous conductive track is provided in proximity to the third cutter region of the needle guard at an end of the extension movement.
   20. The autoinjector of any one of embodiments 16-19, further comprising:
a transfer sleeve disposed within the housing and configured to interact with the plunger rod and the needle guard,
wherein the first continuous conductive track, the second continuous conductive track, and the third continuous conductive track are disposed on a surface of the transfer sleeve.
   21. The autoinjector of any one of embodiments 16-20, further comprising:
a linkage disposed within the housing and configured to interact with the transfer sleeve,
wherein the linkage comprises one or more features (e.g., guard feature and/or latch feature) configured to enable or prevent cutting of the first continuous conductive track, the second continuous conductive track, and/or the third continuous conductive track disposed on the surface of the transfer sleeve based on positions of the plunger rod and/or the needle guard during or at the end of the dispensing movement and/or the extension movement.
   22. The autoinjector of any one of embodiments 0-21, wherein the connectivity region comprises one or more passive electrical components.
   23. The autoinjector of any one of embodiments 0-22, wherein the first sensor comprises a Near Field Communication (NFC) chip, an NFC coil, or an NFC tag.
   24. The autoinjector of embodiment 23, wherein the connectivity region is configured to transmit an electrical signal indicative of said electrical state change from the first sensor to a mobile device.
   25. The autoinjector of any one of embodiments 0-24, further comprising:
an audible clicker configured to produce one or more audible clicks during the dispensing movement of the plunger rod.
   26. The autoinjector of embodiment 25, wherein the audible clicker comprises a ring surrounding the plunger rod, and wherein the ring comprises a deflectable protrusion configured to contact the plunger rod and produce the one or more audible clicks.
   27. The autoinjector of embodiment 26, wherein the plunger rod comprises a ridged surface configured to contact the deflectable protrusion of the audible clicker.
   28. The autoinjector of embodiment 27, wherein each ridge of the ridged surface is configured to cause the deflectable protrusion to deflect during the dispensing movement of the plunger rod.
   29. The autoinjector of any one of embodiments 1-28, wherein the first sensor is configured to detect an ambient temperature in proximity to the container.
   30. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament;
a needle guard configured to retract when an insertion force is applied to insert the needle into a user and/or configured to extend when the insertion force is removed;
a mechanism configured so that a dispensing movement of the plunger rod causes the mechanism to generate a first electrical state change and/or configured so that a retraction movement of the needle guard causes the mechanism to generate a second electrical state change and/or configured so that an extension movement of the needle guard causes the mechanism to generate a third electrical state change; and
a first sensor configured to detect said first, second, and/or third electrical state changes generated by the mechanism.
   31. An autoinjector comprising:
a housing;
a connectivity region configured to be accessed at a region of the housing;
a needle arranged at a distal end of the housing;
a needle guard configured to expose the needle when an insertion force is applied to insert the needle into a user;
a container disposed within the housing and configured to contain medicament;
a plunger slidably disposed within the container;
a plunger rod configured to push the plunger through the container to dispense the medicament through the needle when the container contains medicament and when the needle is exposed; and
a transfer sleeve comprising a plurality of continuous conductive tracks, wherein a movement of the needle guard and/or a movement of the plunger rod generates one or more electrical breaks in at least one of the plurality of continuous conductive tracks.
   32. A system comprising:
an autoinjector of any one of embodiments 1-31; and
a processor configured to process one or more electrical signals to track a dispensing movement of the plunger rod and/or a retraction movement of the needle guard and/or an extension movement of the needle guard.
   33. A method of detecting a dispensing movement of a plunger rod within an autoinjector, the method comprising:
sensing an electrical state change as the plunger rod moves during the dispensing movement, wherein the plunger rod comprises a region configured to generate one or more electrical breaks in a continuous conductive track, and
wherein the dispensing movement of the plunger rod generates the electrical state change.
   34. A method of detecting a retraction movement of a needle guard within an autoinjector, the method comprising:
sensing an electrical state change as the needle guard retracts when an insertion force is applied to insert a needle into a user, wherein the needle guard comprises a region configured to generate one or more electrical breaks in a continuous conductive track, and
wherein the retraction movement of the needle guard generates the electrical state change.
   35. A method of detecting an extension movement of a needle guard within an autoinjector, the method comprising:
sensing an electrical state change as the needle guard extends when an insertion force is removed after inserting a needle into a user, wherein the needle guard comprises a region configured to generate one or more electrical breaks in a continuous conductive track, and
wherein the extension movement of the needle guard generates the electrical state change.
   36. The method of any one of embodiments 33-35, wherein the autoinjector further comprises an audible clicker configured to produce one or more audible clicks during a dispensing movement of a plunger rod of the autoinjector.
   37. The method of embodiment 36, wherein the audible clicker comprises a ring surrounding the plunger rod, and wherein the ring comprises a deflectable protrusion configured to contact the plunger rod and produce the one or more audible clicks.
   38. The method of embodiment 37, wherein the plunger rod comprises a ridged surface configured to contact the deflectable protrusion of the audible clicker.
   39. The method of embodiment 38, wherein each ridge of the ridged surface is configured to cause the deflectable protrusion to deflect during the dispensing movement of the plunger rod.
   40. The method of any one of embodiments 33-39, wherein a first sensor is disposed between a proximal end and a distal end of the housing, and wherein the first sensor is configured to detect the electrical state change generated by the dispensing movement, the retraction movement, and/or the extension movement.
   41. The method of embodiment 40, wherein the first sensor comprises a Near Field Communication (NFC) chip, an NFC coil, or an NFC tag.
   42. The method of embodiment 40 or 41, wherein the first sensor is further configured to detect an ambient temperature in proximity to a container.
   43. The method of any one of embodiments 33-42, further comprising sending, via a Near Field Communication module, signals from a first sensor, if present, to a mobile device.
   44. The method of embodiment 43, further comprising displaying information about the dispensing movement on the mobile device.
   45. The method of any one of embodiments 33-44, further comprising measuring a temperature of medicament within the autoinjector or an ambient temperature within the autoinjector using a temperature sensor.
   46. The method of embodiment 45, further comprising sending, via a Near Field Communication module, signals from the temperature sensor to a mobile device.
   47. The method of embodiment 45 or embodiment 46, further comprising displaying the temperature of the medicament or the ambient temperature on the mobile device; indicating that the temperature of the medicament or the ambient temperature is above a threshold temperature for use of the autoinjector; or both.
   48. A composition comprising lenacapavir or a pharmaceutically accepted salt thereof for use in the prevention or treatment of HIV, wherein the composition is administered via the autoinjector of any of embodiments 1 to 32.
   49. The composition of embodiment 48, wherein the composition is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.
   50. The composition of embodiment 48, wherein the composition is intramuscularly administered via the autoinjector, optionally wherein the composition is a solution comprising about 16.9 w/w % water, about 36.2 w/w % PEG 300, about 41.9 w/w % of lenacapavir sodium, and about 5.0 w/w % ethanol.
   51. Use of lenacapavir or a pharmaceutically accepted salt thereof for the manufacture of a medicament for the prevention or treatment of HIV, wherein the prevention or treatment comprises administering the medicament via the autoinjector of any of embodiments 1 to 32.
   52. The use of embodiment 51, wherein the medicament is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.
   53. The use of embodiment 51, wherein the medicament is intramuscularly administered via the autoinjector, optionally wherein the medicament is a solution comprising about 16.9 w/w % water, about 36.2 w/w % PEG 300, about 41.9 w/w % of lenacapavir sodium, and about 5.0 w/w % ethanol.

1. An autoinjector comprising:
a housing;
a connectivity region configured to be accessed at a region of the housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament;
a needle guard configured to retract when an insertion force is applied to the needle and configured to extend when the insertion force is removed;
wherein a dispensing movement of the plunger rod, a retraction movement of the needle guard, and/or an extension movement of the needle guard generates an electrical state change; and
a first sensor within the connectivity region configured to detect said electrical state change as the plunger rod and/or the needle guard moves.

2. The autoinjector of claim 1, wherein the electrical state change is indicative of dose progression.

3. The autoinjector of claim 1, wherein when the dispensing movement of the plunger rod generates the electrical state change, the electrical state change is configured to indicate completion of the dispensing movement of the plunger rod.

4. (canceled)

5. The autoinjector of claim 1, wherein the medicament comprises lenacapavir or a pharmaceutically accepted salt thereof.

6. The autoinjector of claim 1, wherein the autoinjector further comprises a gas canister assembly configured to release pressurized gas which, when released, provides a force acting on the plunger rod to push the plunger through the container.

7. The autoinjector of claim 1, wherein the plunger rod comprises a first cutter region disposed on a proximal end of the plunger rod, wherein the first cutter region is configured to cut a first continuous conductive track during the dispensing movement of the plunger rod to provide a first non-continuous conductive track.

8-9. (canceled)

10. The autoinjector of claim 7, wherein the needle guard is further configured to expose the needle when the insertion force is applied to insert the needle into a user and during the retraction movement of the needle guard, and wherein the retraction movement of the needle guard generates a second electrical state change arising from a second non-continuous conductive track.

11. The autoinjector of claim 10, wherein the needle guard is further configured to cover the needle when the insertion force is removed to withdraw the needle from the user and during the extension movement of the needle guard, and wherein the extension movement of the needle guard generates a third electrical state change arising from a third non-continuous conductive track.

12. The autoinjector of claim 11, wherein the needle guard comprises a second cutter region and/or a third cutter region.

13. The autoinjector of claim 12, wherein the second cutter region is configured to cut a second continuous conductive track during the retraction movement of the needle guard to provide the second non-continuous conductive track and/or wherein the third cutter region is configured to cut a third continuous conductive track during the extension movement of the needle guard to provide the third non-continuous conductive track.

14. The autoinjector of claim 13, wherein the second continuous conductive track is provided in proximity to the second cutter region of the needle guard at an end of the retraction movement and/or wherein the third continuous conductive track is provided in proximity to the third cutter region of the needle guard at an end of the extension movement.

15. The autoinjector of claim 14, further comprising:
a transfer sleeve disposed within the housing and configured to interact with the plunger rod and the needle guard,
wherein the first continuous conductive track, the second continuous conductive track, and the third continuous conductive track are disposed on a surface of the transfer sleeve.

16. The autoinjector of claim 15, further comprising:
a linkage disposed within the housing and configured to interact with the transfer sleeve,
wherein the linkage comprises one or more features (e.g., guard feature and/or latch feature) configured to enable or prevent cutting of the first continuous conductive track, the second continuous conductive track, and/or the third continuous conductive track disposed on the surface of the transfer sleeve based on positions of the plunger rod and/or the needle guard during or at the end of the dispensing movement and/or the extension movement.

17. (canceled)

18. The autoinjector of claim 1, wherein the first sensor comprises a Near Field Communication (NFC) chip, an NFC coil, or an NFC tag.

19. The autoinjector of claim 18, wherein the connectivity region is configured to transmit an electrical signal indicative of said electrical state change from the first sensor to a mobile device.

20. The autoinjector of claim 0, further comprising:
an audible clicker comprising a deflectable protrusion, the deflectable protrusion configured to contact the plunger rod to produce one or more audible clicks during the dispensing movement of the plunger rod.

21-22. (canceled)

23. The autoinjector of claim 20, wherein the plunger rod comprises a ridged surface configured to cause the deflectable protrusion to deflect during the dispensing movement of the plunger rod.

24. The autoinjector of claim 1, wherein the first sensor is configured to detect an ambient temperature in proximity to the container.

25. (canceled)

26. An autoinjector comprising:
a housing;
a connectivity region configured to be accessed at a region of the housing;
a needle arranged at a distal end of the housing;
a needle guard configured to expose the needle when an insertion force is applied to insert the needle into a user;
a container disposed within the housing and configured to contain medicament;
a plunger slidably disposed within the container;
a plunger rod configured to push the plunger through the container to dispense the medicament through the needle when the container contains medicament and when the needle is exposed; and
a transfer sleeve comprising a plurality of continuous conductive tracks, wherein a movement of the needle guard and/or a movement of the plunger rod generates one or more electrical breaks in at least one of the plurality of continuous conductive tracks.

27. (canceled)

28. A method of detecting movement within an autoinjector, the method comprising:
sensing a first electrical state change as a plunger rod moves during a dispensing movement, wherein the plunger rod comprises a region configured to generate one or more first electrical breaks in a first continuous conductive track, and
wherein the dispensing movement of the plunger rod generates the first electrical state change.

29. The method of claim 28, further comprising:
sensing a second electrical state change as a needle guard retracts when an insertion force is applied to insert a needle into a user, wherein the needle guard comprises a first region configured to generate one or more second electrical breaks in a second continuous conductive track, and
wherein the retraction movement of the needle guard generates the second electrical state change.

30. The method of claim 29, further comprising:
sensing a third electrical state change as the needle guard extends when an insertion force is removed after inserting a needle into the user, wherein the needle guard comprises a second region configured to generate one or more third electrical breaks in a third continuous conductive track, and
wherein the extension movement of the needle guard generates the third electrical state change.

31. The method of claim 28, wherein the autoinjector further comprises an audible clicker comprising a deflectable protrusion, and the method further comprises producing one or more audible clicks during the dispensing movement of the plunger rod of the autoinjector by deflecting the deflectable protrusion with the plunger rod

32-33. (canceled)

34. The method of claim 31, wherein the plunger rod comprises a ridged surface configured to contact the deflectable protrusion to cause the deflectable protrusion to deflect during the dispensing movement of the plunger rod.

35. The method of claim 30, wherein a first sensor is disposed between a proximal end and a distal end of a housing, and wherein the first sensor is configured to detect the first, second, and/or third electrical state change generated by the respective dispensing movement, the retraction movement, and/or the extension movement.

36. The method of claim 35, wherein the first sensor comprises a Near Field Communication (NFC) chip, an NFC coil, or an NFC tag.

37. (canceled)

38. The method of claims 35, further comprising:
sending, via a Near Field Communication module, signals from the first sensor to a mobile device; and
displaying information about the dispensing movement on the mobile device.

39. (canceled)

40. The method of claims 28, further comprising measuring a temperature of medicament within the autoinjector or an ambient temperature within the autoinjector using a temperature sensor.

41. The method of claim 40, further comprising sending, via a Near Field Communication module, signals from the temperature sensor to a mobile device.

42. The method of claim 40, further comprising displaying the temperature of the medicament or the ambient temperature on a mobile device; indicating that the temperature of the medicament or the ambient temperature is above a threshold temperature for use of the autoinjector; or both.

43. A composition comprising lenacapavir or a pharmaceutically accepted salt thereof composition for use in the prevention or treatment of HIV, wherein the composition is administered via the autoinjector of claim 1.

44. Use of lenacapavir or a pharmaceutically accepted salt thereof for the manufacture of a medicament for the prevention or treatment of HIV, wherein the prevention or treatment comprises administering the medicament via the autoinjector of claim 1.
